(19) United States
(12) Patent Application Publication
(10) Pub. No.: US 2016/0000933 A1
(43) Pub. Date: Jan. 7, 2016

(54) CONJUGATED BIOLOGICAL MOLECULES AND THEIR PREPARATION

(71) Applicant: Andrei POLUKHTIN, Scottsdale, AZ (US)

(72) Inventor: Andrei POLUKHTIN, Scottsdale, AZ (US)

(21) Appl. No.: 14/738,566

(22) Filed: Jun. 12, 2015

Related U.S. Application Data
(60) Provisional application No. 61/998,598, filed on Jul. 1, 2014.

(51) Int. Cl. A61K 47/48; A61K 31/704; C07D 257/08; C07C 317/24; C07D 223/24

(52) U.S. Cl. CPC A61K 47/48407; C07C 317/24; C07D 223/24; C07D 257/08; A61K 31/704

(57) ABSTRACT

Conjugation reagents of formula (1) that form a bridge between two cystein residues derived from the disulfide bond and a two-step process for the preparation of antibody conjugates are disclosed.

### BACKGROUND OF THE INVENTION

1. Field of the Invention

This invention relates to a novel process for preparing antibody conjugates and to novel compounds used for the preparation of antibody conjugates. The specificity of antibodies for specific antigens on the surface of target cells and molecules has led to their extensive use as carriers of a variety of diagnostic and therapeutic agents. For example, antibodies conjugated to labels and reporter groups, such as fluorescent dyes, radioisotopes and enzymes find use in labeling and imaging applications, while conjugation to cytotoxic agents and chemotherapy drugs allows targeted delivery of such agents to specific tissues or structures, for example particular cell types or growth factors, minimizing the impact on normal, healthy tissue and significantly reducing the side effects associated with chemotherapy treatments. Antibody-drug conjugates have extensive potential therapeutic applications in several disease areas, particularly in cancer.

2. Description of the Background

Water soluble, synthetic polymers, particularly polyalkylene glycols, are widely used to conjugate therapeutically active molecules, such as proteins. These therapeutic conjugates have been shown to alter pharmacokinetics favorably by prolonging circulation time and decreasing clearance rates, decreasing systemic toxicity, and in several cases, displaying increased clinical efficacy. The process of covalently conjugating polyethylene glycol, PEG, to proteins is commonly known as “PEGylation.”

Many small molecule or polymer reagents for conjugation comprise conjugating chemical functionality that is hydrolytically unstable. Examples of hydrolytically unstable conjugating reagents are active esters that include, for example, polyalkylene oxide-N-succinimide carbonates (U.S. Pat. No. 5,122,614). These reagents have relatively short half lives in aqueous media. This results in the need to add large stoichiometric excesses of the conjugating reagent. The hydrolytic stability of the reagent is important because the requirement to add stoichiometric excesses for protein conjugation requires significant effort and cost to purify the polymer-protein conjugate from the reaction mixture. Furthermore, these hydrolytically unstable reagents tend to undergo, preferentially, a reaction with amine chemical functionality in the protein, particularly to the ε-amine of lysine residues. Since most proteins of interest have more than one lysine residue, and frequently many lysine residues, conjugation tends to be non-specific in that it occurs at many residue sites on the protein. It is possible to purify the conjugating reaction mixture to isolate proteins conjugated to one polymer molecule; however, it is not possible to isolate, at a reasonable cost, polymer-protein conjugates that are all conjugated to the same amine group on the protein. Non-specific conjugation frequently results in impaired protein function. For example, antibodies and antibody fragments with random poly(alkylene oxide) attachment via lysine residues result in modified antibodies (or modified antibody fragments) able to bind target antigen with reduced affinity, avidity or specificity. Additionally, amine specific polymer conjugating reagents require conjugating reaction conditions that must be selected to ensure that the amines on the protein are not protonated. These conditions require moderately high pH media (7-9), which allows the amine moieties to be reactive enough to react with the conjugating reagent. High pH conditions might be deleterious to the protein, causing structural changes and denaturation. These processes result in impairment of protein function. Amine specific conjugation reagents tend to bind to accessible amine sites on the protein. These reagents can be termed kinetic reagents. They are labile and undergo a reaction with the most assessable amino nucleophilic sites on the protein. Amine specific polymer conjugating reagents that conjugate by amine acylation result in the loss of positive charge on the amine group of the amino acid residue on the protein that would normally be present under physiological conditions for the unconjugated protein. These features of the amine specific polymer conjugating reagents often lead to partial impairment of the function of the protein. Other conjugating functional groups incorporated in polymers for conjugation to protein and that are amine specific and frequently hydrolytically labile include isocyanate (WO 94/04193) and carbonates (WO 90/13540).

Particularly relevant for optimized efficacy and to ensure dose to dose consistency is to make certain that the number of conjugated polymer molecules per protein is the same and that each payload molecule is specifically covalently conjugated to the same amino acid residue in each protein molecule. Non-specific conjugation at sites along a protein molecule results in a distribution of conjugation products, and frequently unconjugated protein, to give a complex mixture that is difficult, tedious, and expensive to purify.

Thiol specific conjugating reagents for proteins have been developed to address the limitations for the propensity of the conjugating reagent to undergo hydrolysis that is competitive with conjugation to the protein, non-specific polymer conjugation at different amino acid residues in the protein and the need for high pH conjugating reaction conditions. Thiol specific polymer conjugating reagents can be utilized at pH values close to neutral where the amine functional moieties on the amino acid residues of the protein are protonated and thus cannot effectively compete in the conjugation reaction with the conjugating reagent. Thiol specific conjugating reagents that are relatively more hydrolytically stable than are the aforementioned amine specific reagents can be utilized at lower stoichiometric excess, thus reducing the cost during purification of the protein conjugate. Conjugating functional moieties that are broadly selective for thiol groups include iodoacetamide, maleiimide (WO 92/16221), vinylsulfone (WO 95/13312 and WO 95/34326), vinyl pyridines (WO 88/05433), and acrylate and methacrylate esters (WO 99/01469). These thiols selective conjugating moieties yield a single thioether conjugating bond between the polymer.

Most proteins do not have free sulfhydryls because these sulfhydryls undergo rearrangement and scrambling reactions with the disulfide bridges within the protein, resulting in impaired protein function. For proteins that do have free sulfhydryls, these sulfhydryls are frequently critical for protein function. Typically in a protein, the number of sulfhydryl moieties is less than the number of amine moieties (e.g., lysine or histadine). Since conjugation to a protein can be made to be specific at thiol groups and since proteins do not typically have free thiol groups, site-specific modification of protein by mutagenesis can be used to introduce thiol sites for PEG attachment. However, such modifications increase costs significantly. The introduced free sulfhydryl can have similar limitations as mentioned heretofore in the engineered protein for protein scrambling and protein dimerization. Also, the process of mutagenesis and production of the modified protein from bacterial sources frequently causes the free sulfhydryl to be bound in a disulfide bond with glutathione, for example. Interleukin-2, for example, has been modified by mutagenesis to replace a threonine residue by a cysteine to allow site specific attachment of PEG. [Goodson R J, Katre N V; Bio/Technology (1990) 8, 343-346].

It is known in the art that conjugating parameters have to be optimally matched with the therapeutically active molecule of interest in terms of polymer morphology, molecular weight characteristics, and chemical functionality. Although the polymer protein conjugate can display many favorable and necessary properties needed for safe, effective medical use, the effect of polymer conjugation on the activity and stability of the protein is of vital importance for performance. Conjugation variables related to the location and amount of conjugation and polymer characteristics must be optimally correlated with biological and physicochemical properties.

Rosario et al., Bioconjugate Chemistry 1 (1990) 51-59, and Liberatore et al., Bioconjugate Chemistry 1 (1990) 36-50 describe a series of novel, bis-sulfone based conjugation reagents, which can be used to conjugate with both sulfur atoms derived from two cysteine residues in a protein to give novel thioether conjugates. The same authors showed that an α-methylene leaving group and a double bond are cross-conjugated with an electron withdrawing function that serves as a Michael activating moiety. If the leaving group is prone to elimination in the cross-functional reagent rather than to direct displacement and the electron-withdrawing group is a suitable activating moiety for the Michael reaction, then sequential intramolecular bis-alkylation can occur by consecutive Michael and retro Michael reactions. The leaving moiety serves to mask a latent conjugated double bond that is not exposed until after the first alkylation has occurred, and bis-alkylation results from sequential and interactive Michael and retro-Michael reactions as described in J. Am. Chem. Soc. 1979, 101, 3098-3110 and J. Am. Chem. Soc. 1988, 110, 5211-5212). The electron withdrawing group and the leaving group are optimally selected, so bis-alkylation can occur by sequential Michael and retro-Michael reactions.

The major shortfall of the bis-alkylation reagents described above is a low cross-linking efficiency as well as a low efficiency of labeling. Rosario et al., Bioconjugate Chemistry 1 (1990) 51-59 and Liberatore et al., Bioconjugate Chemistry 1 (1990) 36-50 describe labeling experiments with several bis-alkylation reagents leading to a typical cross-linking efficiency of 15 to 30% along with only a single example of efficiency reaching 40%. Even through bis-sulfone based conjugation reagents has been known for more than 20 years, there was only one example of its efficient application to an intact protein-small molecule (Badescu et al., Bioconjugate Chemistry 25 (2014) 1124). However, a long polyethylene glycol linker must be incorporated between a bis-alkylating moiety and a payload moiety. In some applications, the incorporation of a long linker might be acceptable; however, in other applications, it might not be desirable or even acceptable. In addition, the incorporation of a long polyethylene glycol linker between a bis-alkylating moiety and a payload might be a synthetically challenging task.

U.S. Ser. No. 13/919,217 describes a series of bis-sulfone based conjugation reagents, which can be used to react with nucleophilic groups in a protein to produce a protein-polymer conjugate. These reagents find particular utility for their ability to conjugate with both sulfur atoms derived from a disulfide bond in a protein to give thioether conjugates; however, the labeling efficiency still remains very low at 10-26% (Example 2, human IgG, Fab fragment), and the best example shows labeling efficiency of 40% after incubation for 20 hours with a significant excess of bis-alkylating reagent (Example 6). These results emphasize that there is an urgent need for efficient bis-alkylating reagents and methods for conjugation with both sulfur atoms derived from a disulfide bond in a protein to give thioether conjugates.

As such, the present invention is directed to a series of novel reagents and methods, which can be used to conjugate with both sulfur atoms derived from two cysteine residues in a protein to give novel thioether conjugates. The invention in the first instance is intended for the conjugation of the two sulfur atoms that form natural disulphide bridges in native proteins. Disulfide bonds are found in medically relevant proteins, specifically, secretory proteins, lysosomal proteins, and the exoplasmic domains of membrane proteins. The technology provides clear advantages over known techniques for conjugating small molecules and polymers to proteins while preserving the tertiary structure of the proteins.

### SUMMARY OF THE INVENTION

This invention provides novel bis-alkylating reagents for preparing antibody conjugates via conjugation through both sulfur atoms derived from two cysteine residues in a protein. The present invention also provides methods using bioorthogonal chemistry to cross-link and functionalize antibodies or its Fab fragments with difficult substrates. The invention arises from the finding that incorporation of a charged moiety, such as a sulfo group (SO₃H), a mid-length hydrophilic moiety, such as mPEG23, or a combination of both between a bis-sulfone moiety and a payload not only increases solubility in an aqueous solution, but also dramatically increases the cross-linking and labeling efficiency.

In addition to improved linkers for direct (one-step) conjugation, a strategy was developed based on a two-step approach. Improved linkers were developed, which contain (i) a charged moiety or a mid-sized mPEG moiety or a combination of both and (ii) a bioorthogonal group, that are capable of efficiently cross-linking and modifying an antibody or antibody fragment, followed by reacting with a reaction partner that contains the moiety of interest (FIG. 3). Linkers were also developed that included a spacer and can be used for conjugation of small molecules. Different linkers were used to demonstrate the concept. This two-step approach permitted significantly higher completion of coupling in a composition of antibodies. In one advantageous configuration, a trans-cycloocten reactive group was placed on a linker comprising a spacer, and a tetrazine group (complementary reactive group) was placed on the reaction partner. The two-step approach permitted the completion of coupling of larger and/or hydrophobic moieties.

The two-step approach displays advantages that permitted preparation of compounds that have moieties that are subject to degradation when maintained at 37° C., the temperature at which the cross-linking is the most efficient. The two-step approach also permitted the preparation of conjugates using lower equivalents of complementary reactive moiety (compared to antibodies). While direct coupling required several equivalents of bis-sulfone-payload substrate for significant coupling (to the extent that this was even possible), the multi-step approach decreased the number of equivalents of complementary reactive moiety needed to slightly above one equivalent. For compounds such as hydrophobic compounds and large organic molecules such as toxins, high concentrations can be problematic and, moreover, often require organic solvents, which result in the aggregation of antibodies.

In a first embodiment, the present invention is directed to a compound of the general formula (1):

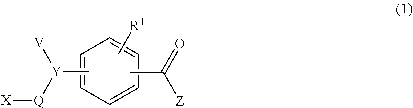

wherein
X represents a payload;
Q represents a cleavable or non-cleavable linking group;
Y represents an amide group;
V represents a moiety that improves cross-linking and/or labeling efficiency;
Z represents either —CH—(CH₂L)₂ or —C(CH₂L)(═CH₂), L independently represents a leaving group, and
R¹ represents H, alkyl, —CN, —NO₂, CO₂R, —COH, CH₂OH, COR², —OR², —OCOR², —OCO₂R², —SR², SO, —SO₂R², —NHCOR², —NR²COR², NHCO₂R², —NO, —NHOH, —NR²OH, —C═N—NH—COR², halogen, C≡CR², —C═CR²₂, or —C═CHR, in which each R² independently represents a hydrogen atom or alkyl, aryl, or alkyl-aryl group.

In the general formula, L may represent —SR³, —SO₂R³, —OSO₂R³, —N⁺R³₃, —N⁺HR³₂, —N⁺H₂R³, halogen, or —O◯, in which R³ represents a hydrogen atom or an alkyl, aryl, or alkyl-aryl, and ◯ represents a substituted aryl group containing at least one electron withdrawing substituent. V may represent —(CH₂)ₙSO₃X, in which n=1-6 and X represents H or counterion. V may represent a hydrophilic polymer. X may represent a drug, a diagnostic moiety, or a chelating agent. X may represent a detection moiety, such as a fluorescent compound, or hapten. X may represent a protein.

In a second embodiment, the present invention is directed to a kit comprising at least one compound described above in the first embodiment of the present invention.

In a third embodiment, the present invention is directed to a protein conjugate of the general formula (2):

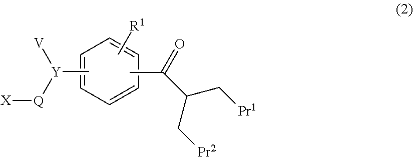

in which X, Q, Y and V have the meanings given above, and each of Pr₁ and Pr₂ independently represents a separate protein or peptide molecule, or Pr₁ and Pr₂ together represent a single protein or peptide Pr bonded at two separate points to obtain the following formula (IIa):

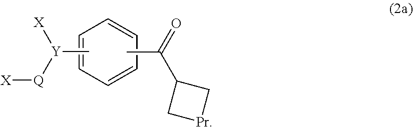

Preferably, Pr₁ and Pr₂ together represent a single protein bonded to two sulfur atoms derived from a disulfide bond in a protein or to two histidine residues present in a polyhistidine tag attached to a single protein.

In a fourth embodiment, the present invention is directed to a compound of the general formula (3):

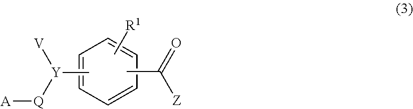

wherein:
Q represents a cleavable or non-cleavable linking group;
Y represents an amide group;
V represents a moiety that improves cross-linking and/or labeling efficiency or is absent;
Z represents either —CH—(CH₂L)₂ or —C(CH₂L)(═CH₂);
L independently represents a leaving group;
R¹ represents H, alkyl, —CN, —NO₂, CO₂R, —COH, —CH₂OH, —COR², —OR², —OCOR², —OCO₂R², —SR², —SO, —SO₂R², —NHCOR², —NR²COR², —NHCO₂R², —NO, —NHOH, —NR²OH, —C═N—NH—COR², halogen, —C≡CR², —C═CR²₂, or —C═CHR, in which each R² independently represents a hydrogen atom or alkyl, aryl, or alkyl-aryl group; and A represents a reactive moiety partner of a pair of orthogonally reactive moieties that can react with each other without activation and both reactive moieties are sufficiently stable under commonly applied protein labeling conditions.

In the general formula, L may represent —SR³, —SO₂R³, —OSO₂R³, —N⁺R³₃, —N⁺HR³₂, —N⁺H₂R³, halogen, or —O◯, in which R³ represents a hydrogen atom or an alkyl, aryl, or alkyl-aryl, and ◯ represents a substituted aryl group containing at least one electron withdrawing substituent. V may represent H. V may represent —(CH₂)ₙSO₃X, in which n=1-6 and X represents H or counterion. V may represent a hydrophilic polymer. A may be selected from the group consisting of orthogonal reactive pairs that undergo Staudinger ligation, strain-promoted Huisgen 1,3-cycloaddition, Inverse Demand Diels-Alder cycloaddition, and hydrazone or oxime bond forming reactions. A may represent a 1,3-dipole group for example, azide, nitrile oxide, nitrone, azoxy group, and acyl diazo group. A may represent a substituted or unsubstituted cyclooctyne that undergoes a 1,3-cycloadditon reaction with a 1,3 dipole group. A may represent a diene that undergoes an Inverse electron-demand Diels-Alder reaction, for example a substituted or unsubstituted tetrazine. A may represent a dienophile that undergoes Inverse electron-demand Diels-Alder reaction, for example a substituted or unsubstituted trans-cyclooctene.

In a fifth embodiment, the present invention is directed to a process for the preparation of a protein conjugate of the general formula (4):

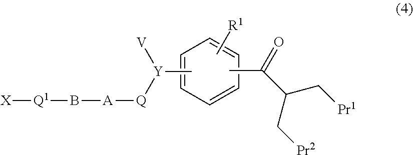

wherein:
X represents a payload;
Q and Q¹ independently represent a cleavable or non-cleavable linking group;
Y represents an amide group;
V represents a moiety that improves cross-linking and/or labeling efficiency or is absent;
R¹ represents H, alkyl, —CN, —NO₂, —CO₂R, —COH, —CH₂OH, —COR², —OR², —OCOR², —OCO₂R², —SR², —SO, —SO₂R², —NHCOR², —NR²COR², —NHCO₂R², —NO, —NHOH, —NR²OH, —C═N—NH—COR², halogen, —C≡CR², —C═CR²₂, or —C═CHR, in which each R² independently represents a hydrogen atom or alkyl, aryl, or alkyl-aryl group;
both Pr¹ or Pr² together represent a single protein or peptide bonded at two separate points via two thiol groups generated by the reduction of a disulfide bridge in the protein;
A-B is a pair of orthogonally reactive moieties that can react with each other without activation and are stable under commonly applied protein labeling conditions;
said method comprising the steps of:
(a) reducing a disulfide bridge in the protein; and
(b) reacting the reduced protein with a compound of general formula (3) to form an activated protein of general formula (5):

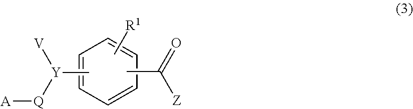

wherein Z represents either —CH—(CH₂L)₂ or —C(CH₂L)(═CH₂), L independently represents a leaving group;

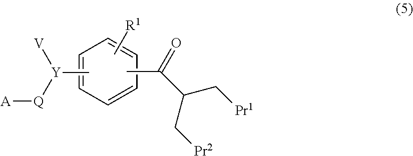

(c) reacting said activated protein with a compound of general formula (6):

X-Q¹-B (6).

In the method, L may represent —SR³, —SO₂R³, —OSO₂R³, —N⁺R³₃, —N⁺HR³₂, —N⁺H₂R³, halogen, or —O◯, in which R³ represents a hydrogen atom or an alkyl, aryl, or alkyl-aryl, and ◯ represents a substituted aryl group containing at least one electron withdrawing substituent. V may represent H. V may represent —(CH₂)ₙSO₃X, in which n=1-6 and X represents H or counterion. V may represent a hydrophilic polymer. A may be selected from the group consisting of orthogonal reactive pairs that undergo Staudinger ligation, strain-promoted Huisgen 1,3-cycloaddition, Inverse Demand Diels-Alder cycloaddition, and hydrazone or oxime bond forming reactions. A may represent a 1,3-dipole group for example, azide, nitrile oxide, nitrone, azoxy group, and acyl diazo group. A may represent a substituted or unsubstituted cyclooctyne that undergoes a 1,3-cycloadditon reaction with a 1,3 dipole group. A may represent a diene that undergoes an Inverse electron-demand Diels-Alder reaction, for example a substituted or unsubstituted tetrazine. A may represent a dienophile that undergoes an Inverse electron-demand Diels-Alder reaction, for example a substituted or unsubstituted trans-cyclooctene. X may represent a drug, a diagnostic moiety, or a chelating agent. X may represent a biopolymer such as protein or a synthetic polymer such as PEG.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to one of ordinary skill in the art from this detailed description.

### BRIEF DESCRIPTION OF THE DRAWINGS

The present invention will become more fully understood from the detailed description given below and the accompanying drawings that are given by way of illustration only and are thus not limitative of the present invention.

FIG. 1 illustrates the mechanism of action of bis-sulfone based conjugation reagents.

FIG. 2 illustrates the single step labeling approach using bis-sulfone based conjugation reagents.

FIG. 3 illustrates two step labeling approach using bis-sulfone based conjugation reagents.

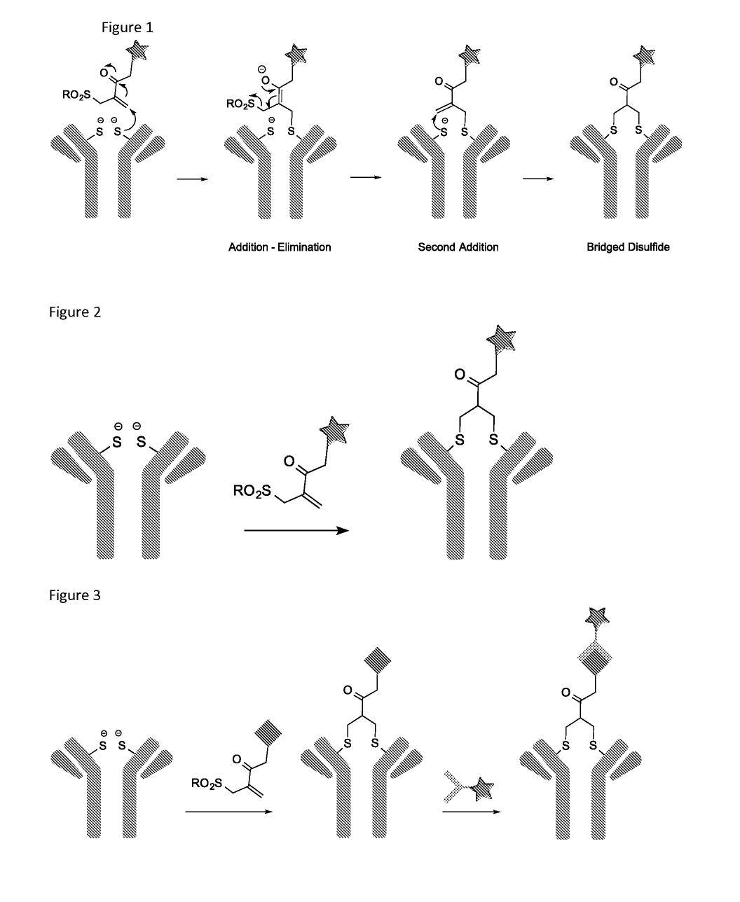

### DETAILED DESCRIPTION OF THE INVENTION

The present invention will now be described with reference to the accompanying drawing.

Unless defined otherwise, all terms used herein have the same meaning as is commonly understood by one of ordinary skill in the art to which this invention belongs. All patents, patent applications, and publications referred to throughout the disclosure herein are incorporated by reference in their entirety. In the event that there is a plurality of definitions for a term herein, those definitions in this section prevail.

As used in the specification, “a” or “an” may mean one or more. As used in the claims, when used in conjunction with the word “comprising,” the words “a” or “an” may mean one or more than one. As used herein, “another” may mean at least a second or more.

Where “comprising” is used, this can be replaced by “consisting essentially of” or by “consisting of.”

The term “antibody” herein is used in the broadest sense and specifically includes full-length monoclonal antibodies, polyclonal antibodies, multispecific antibodies (e.g., bispecific antibodies), and antibody fragments so long as they exhibit the desired biological activity. Various techniques relevant to the production of antibodies are provided in, e.g., Harlow, et al., ANTIBODIES: A LABORATORY MANUAL, Cold Spring Harbor Laboratory Press, Cold Spring Harbor, N.Y., (1988).

An “antibody fragment” comprises a portion of a full-length antibody, preferably an antigen-binding portion or variable regions thereof. Examples of antibody fragments include Fab, Fab′, F(ab)₂, F(ab′)₂, F(ab)₃, Fv (typically the VL and VH domains of a single arm of an antibody), single-chain Fv (scFv), dsFv, Fd fragments (typically the VH and CH1 domain), and dAb (typically a VH domain) fragments; VH, VL, VhH, and V-NAR domains; minibodies, diabodies, triabodies, tetrabodies, and kappa bodies (see, e.g., Ill et al., Protein Eng 1997; 10: 949-57); camel IgG; IgNAR; and multispecific antibody fragments formed from antibody fragments, and one or more isolated CDRs or a functional paratope, where isolated CDRs or antigen-binding residues or polypeptides can be associated or linked together so as to form a functional antibody fragment. Various types of antibody fragments have been described or reviewed in, e.g., Holliger and Hudson, Nat Biotechnol 2005; 23, 1126-1136; WO2005040219; and published U.S. Patent Applications 2005/0238646 and 2002/0161201.

“Fab” or “Fab region” as used herein means the polypeptide that comprises the VH, CH1, VL, and CL immunoglobulin domains. Fab may refer to this region in isolation or this region in the context of a full length antibody, antibody fragment or Fab fusion protein or any other antibody embodiments as outlined herein.

“Fv” or “Fv fragment” or “Fv region” as used herein means a polypeptide that comprises the VL and VH domains of a single antibody.

“Fc,” “Fc domain,” or “Fc region” as used herein means the polypeptide comprising the constant region of an antibody, excluding the first constant region immunoglobulin domain. Thus, Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain.

An “isolated” molecule is a molecule that is the predominant species in the composition wherein it is found with respect to the class of molecules to which it belongs (i.e., it makes up at least about 50% of the type of molecule in the composition and typically will make up at least about 70%, at least about 80%, at least about 85%, at least about 90%, at least about 95%, or more of the species of the molecule (e.g., a peptide) in the composition). Commonly, a composition of an antibody molecule will exhibit 98%, 98%, or 99% homogeneity for antibody molecules in the context of all present peptide species in the composition or at least with respect to substantially active peptide species in the context of proposed use.

The term “reactive moiety” or “reactive group” herein refers to a moiety that can be coupled with another moiety without prior activation or transformation.

The term “bioorthogonal chemistry” refers to any chemical reaction that can occur in the presence of rich functionalities of biological media without interfering with native biochemical processes.

The term “protecting group” refers to a group that temporarily protects or blocks (i.e., is intended to prevent from reacting) a functional group (e.g., an amino group, a hydroxyl group, or a carboxyl group) during the transformation of a first molecule to a second molecule.

The phrase “moiety that improves cross-linking and labeling” when referring to a compound refers to a moiety that changes the cross-linking and labeling properties of the compound in such a way that a better cross-linking and labeling efficiency can be obtained.

The phrase “linking group” refers to a structural element of a compound that links one structural element of said compound to one or more other structural elements of said same compound.

As used herein, “alkyl” refers to a straight or branched hydrocarbon chain that comprises a fully saturated (no double or triple bonds) hydrocarbon group. The alkyl group may have, for example, 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group of the compounds may be designated as “C1-C4 alkyl” or similar designations. By way of example only, “C1-C4 alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted.

As used herein, the term “heteroalkyl” refers to a straight or branched alkyl group that contains one or more heteroatoms, that is, an element other than carbon (including but not limited to oxygen, sulfur, nitrogen, and phosphorus) in place of one or more carbon atoms.

Whenever a group is described as being “substituted,” the group is substituted with one or more of the indicated substituents. If no substituents are indicated, it is meant that the indicated “substituted” group may be substituted with one or more group(s) individually and independently selected from alkyl, alkenyl, alkynyl, cycloalkyl, cycloalkenyl, cycloalkynyl, heteroalkyl, aryl, heteroaryl, heteroalicyclyl, aralkyl, heteroaralkyl, (heteroalicyclyl)alkyl, hydroxy, alkoxy, aryloxy, acyl, mercapto, alkylthio, arylthio, cyano, halogen, thiocarbonyl, carbamyl, thiocarbamyl, amido, sulfonamido, carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, an amino, a mono-substituted amino group and a di-substituted amino group, and protected derivatives thereof.

Where the number of substituents is not specified (e.g., haloalkyl), there may be one or more substituents present. For example, “haloalkyl” may include one or more of the same or different halogens. As another example, “C1-C3 alkoxyphenyl” may include one or more of the same or different alkoxy groups containing one, two or three atoms.

Accordingly, the present invention provides a compound of the general formula (1):

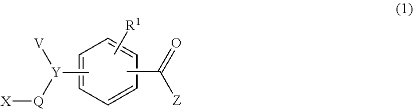

in which each X represents a small molecule payload or bioorthogonal reactive moiety such as trans-cycloocten, tetrazine, cyclooctyne, alkyne, or azide; Q represents a linker; Y represents an amide group; V represent a moiety that improves cross-linking and labeling efficiency; Z represents either —CH(CH₂L)₂ or —C(CH₂L)(═CH₂), in which each L independently represents a leaving group; and R¹ represents H, alkyl, —CN, —NO₂, CO₂R, —COH, CH₂OH, COR², —OR², —OCOR², —OCO₂R², —SR², SO, —SO₂R², —NHCOR², —NR²COR², NHCO₂R², —NO, —NHOH, —NR²OH, —C═N—NH—COR², halogen, C≡CR², —C═CR²₂, or —C═CHR, in which each 1 independently represents a hydrogen atom or alkyl, aryl, or alkyl-aryl group.

The reagents of the formula (1) contain at least one payload or bioorthogonal reactive group. Each payload X may, for example, be a toxin such as duocarmycins, maytansanoids, alkylating agents, taxanes, auristatins, a diagnostic moiety such as radioiodine, or a chelating agent such as DOTA. Exemplary bioorthogonal reactive moieties are trans-cycloocten, tetrazine, cyclooctyne, or azide. Preferably, X may be a drug, a diagnostic moiety, or a chelating agent. More preferably, X may be a detection moiety, such as a fluorescent compound, or hapten. Even more preferably, X may be a protein.

The linkage via the linker Q to the X may be by way of a hydrolytically labile bond or by a non-labile bond. The exact nature of the linker Q depends on a particular application. In some applications, long PEG linkers are beneficial, and in other applications, short linkers such as —(CH₂)ₙ— might be more suitable. Q may be a cleavable or non-cleavable linking group.

A moiety that improves cross-linking and labeling efficiency might be a charged group attached directly to Y or through a short linker. V may be, for example, —(CH₂)ₙ—SO₃H, where n=3, 4, 5, or 6. Another example of a moiety that improves cross-linking and labeling efficiency is a mid-sized hydrophilic polymer moiety such as mPEG11 attached directly to Y. Yet another example of such a moiety is a combination of a charged group and a mid-sized hydrophilic polymer. V may also be —(CH₂)ₙ—SO₃X wherein n is 1-6 and X is H or a counterion. V may also be a hydrophilic polymer.

The improved cross-linking efficiency in the case of a charged group attached directly to Y or through a short linker, such as —(CH₂)ₙ—SO₃H, presumably arises from repulsions between negatively charged —SO₃H groups that prevent stacking or being in close proximity of two hydrophobic bis-sulfone molecules in aqueous media, allowing only one molecule to reach two thiols of a reduced disulfide bond and react with these thiols. The rate of cross-linking depends on the concentration of both reagents, a reduced antibody or its fragment, and the concentration of a bis-sulfone. In order to run the cross-linking process to completion in a reasonable amount of time, a several fold excess of bis-sulfone should be used, requiring higher concentrations of this reagent. An increase in the concentration of bis-sulfone reagent in aqueous media most likely results in a substantial increase of dimer formation through π-π stacking or aggregation, a well know phenomena responsible for the formation of a dimer and the loss of fluorescence in aqueous media for many fluorescent dyes. Formation of either dimer or aggregates most likely will have a negative effect on cross-linking efficiency since it results in two bis-sulfone molecules reacting with both sulfur atoms derived from the disulfide bridge.

The incorporation of a mid-sized hydrophilic polymer, such mPEG12, prevents or at least reduces the formation of dimers and aggregates in aqueous media, presumably by creating bulk surrounding the bis-sulfone moiety.

A leaving group L may, for example, represent —SR, —SO₂R, —OSO₂R, —N⁺R₃, —N⁺HR₂, —NH₂R, halogen, or —O◯, in which R represents a hydrogen atom or an alkyl, aryl or alkyl-aryl group, and ◯ represents a substituted aryl, especially a phenyl group, containing at least one electron withdrawing substituent, for example —CN, —NO₂, —CO₂R, —COH, —CH₂OH, —COR, —OR, —OCOR, —OCO₂R, —SR, —SOR, —SO₂R, —NHCOR, —NRCOR, —NHCO₂R, —NRCO₂R, —NO, —NHOH, —NROH, —C═N—NHCOR, —C═N—NRCOR, —N⁺ R₃, —N⁺HR₂, —NH₂R, halogen (for example, chlorine or, especially, bromine or iodine), C≡CR, —C═CR₂ and —C═CHR, in which each R independently has one of the meanings given above. Alkyl or aryl sulfonyl groups are particularly preferred leaving groups, with phenylsulfonyl or, especially, tosyl, being especially preferred. Where two Ls are present, these may be different groups, but preferably they are the same group.

Except where otherwise stated, substituents which may be present on any optionally substituted aryl, for example phenyl, or heteroaryl group present in a compound of formula (1) include, for example, one or more of the same or different substituents selected from alkyl (preferably C1-4 alkyl, especially methyl, optionally substituted by OH or CO₂H), —CN, —NO₂, —CO₂R, —COH, —CH₂OH, —COR, —OR, —OCOR, —OCO₂R, —SR, —SOR, —SO₂R, —NHCOR, —NHCO₂R, —NO, —NHOH, —NROH, —C═N—NHCOR, —N⁺R₃, —NH₃, —N⁺HR₂, —NH₂R, halogen (for example, fluorine or chlorine), —C≡CR, —C═CR₂ and —C═CHR, in which each R independently has one of the meanings given above. Preferred substituents, if present, include, for example, CN, NO₂, —OR, —OCOR, —SR, —NHCOR, —NHOH and —NRCOR.

R¹ is preferably hydrogen.

In some embodiments, reagents according to the invention have the formula:

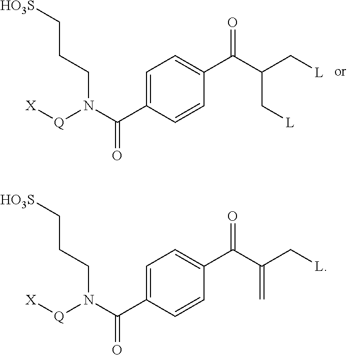

In these reagents, X is preferably a payload; and Q is a linker connecting a payload to an amide group. In addition, in these reagents, each L is preferably a tosyl group as shown in the formulas below:

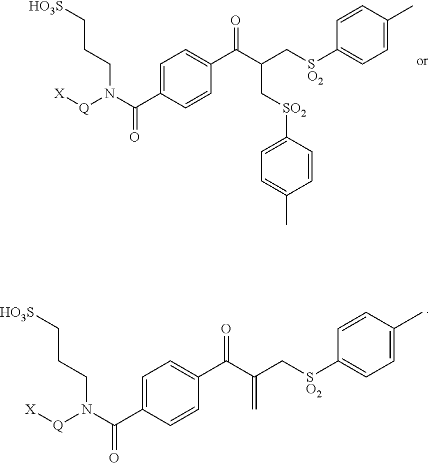

In yet another embodiment, reagents according to the invention have the formula:

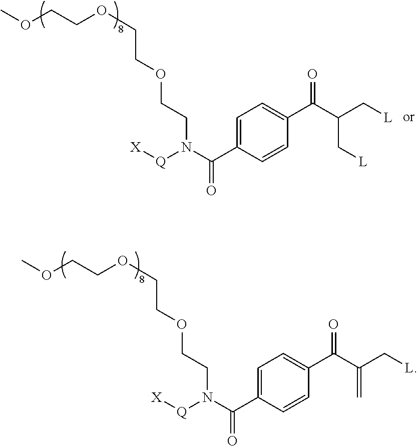

In these reagents, X is preferably a payload; and Q is a linker connecting a payload to an amide group. In addition, in these reagents, each L is preferably a tosyl group as shown in the formulas below:

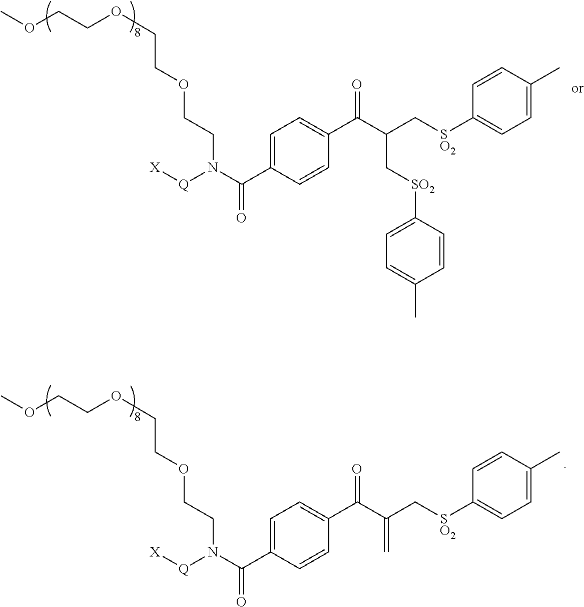

In yet another embodiment, reagents according to the invention have the formula:

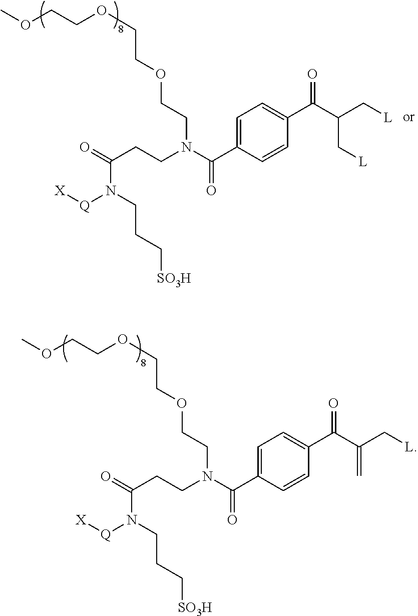

In these reagents, X is preferably a payload; and Q is a linker connecting a payload to an amide group. In addition, in these reagents, each L is preferably a tosyl group as shown in the formulas below:

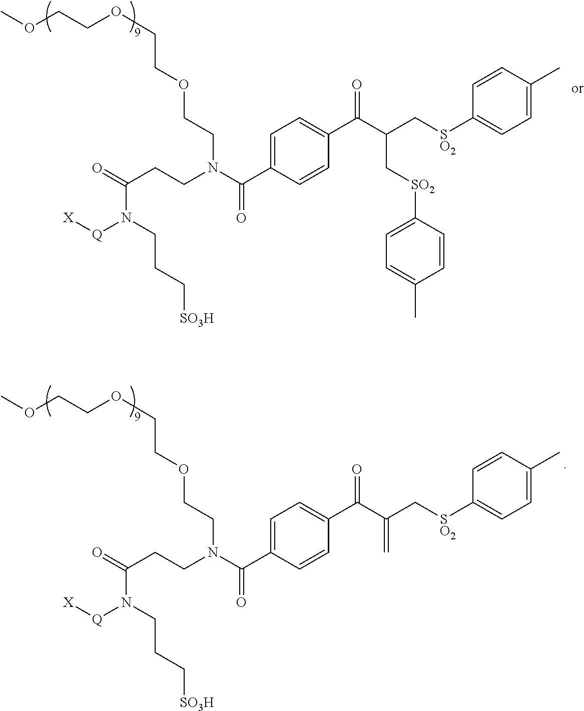

In yet another embodiment, reagents according to the invention have the formula:

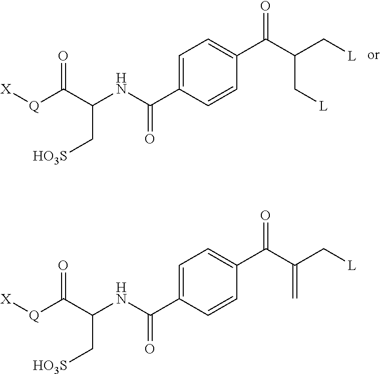

In these reagents, X is preferably a payload; and Q is a linker connecting a payload to a carbonyl group. In addition, in these reagents, each L is preferably a tosyl group as shown in the formulas below:

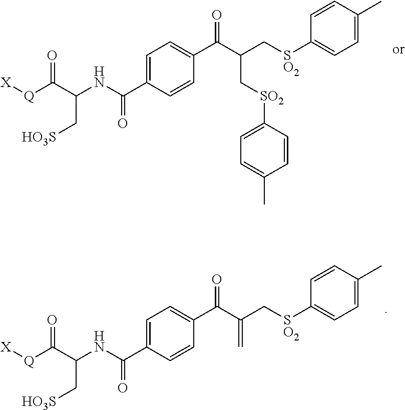

The above compounds of formula (1) may also be part of a kit.

In another embodiment, the present invention is directed to a compound of the general formula (3):

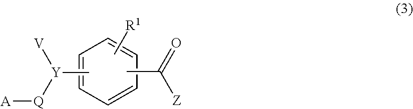

wherein:
Q represents a cleavable or non-cleavable linking group;
Y represents an amide group;
V represents a moiety that improves cross-linking and/or labeling efficiency or is absent;
Z represents either —CH—(CH₂L)₂ or —C(CH₂L)(═CH₂);
L independently represents a leaving group;
R¹ represents H, alkyl, —CN, —NO₂, CO₂R, —COH, —CH₂OH, —COR², —OR², —OCOR², —OCO₂R², —SR², —SO, —SO₂R², —NHCOR², —NR²COR², —NHCO₂R², —NO, —NHOH, —NR²OH, —C═N—NH—COR², halogen, —C═CR², —C═CR²₂, or —C═CHR, in which each R² independently represents a hydrogen atom or alkyl, aryl, or alkyl-aryl group; and
A represents a reactive moiety partner of a pair of orthogonally reactive moieties that can react with each other without activation and both reactive moieties are sufficiently stable under commonly applied protein labeling conditions.

In the general formula, L may represent —SR³, —SO₂R³, —OSO₂R³, —N⁺R³₃, —N⁺HR³₂, —N⁺H₂R³, halogen, or —O◯, in which R³ represents a hydrogen atom or an alkyl, aryl, or alkyl-aryl, and ◯ represents a substituted aryl group containing at least one electron withdrawing substituent.

V may represent H. V may represent —(CH₂)ₙSO₃X, in which n=1-6 and X represents H or counterion. V may represent a hydrophilic polymer.

A may be selected from the group consisting of orthogonal reactive pairs that undergo Staudinger ligation, strain-promoted Huisgen 1,3-cycloaddition, Inverse Demand Diels-Alder cycloaddition, and hydrazone or oxime bond forming reactions. A may represent a 1,3-dipole group for example, azide, nitrile oxide, nitrone, azoxy group, and acyl diazo group. A may represent a substituted or unsubstituted cyclooctyne that undergoes a 1,3-cycloadditon reaction with a 1,3 dipole group. A may represent a diene that undergoes an Inverse electron-demand Diels-Alder reaction, for example a substituted or unsubstituted tetrazine. A may represent a dienophile that undergoes Inverse electron-demand Diels-Alder reaction, for example a substituted or unsubstituted trans-cyclooctene.

Q, Y, V, Z, and R¹ may also be defined the same as in formula (1) above.

The compounds of formula (1) may be used for conjugation to a protein or peptide. For convenience, the term “protein” will be used throughout this Specification, and except where the context requires otherwise, the use of the term “protein” should be understood to include a reference to a peptide.

Accordingly, the invention further provides a process for the preparation of a conjugate, which comprises reacting a compound of the general formula (1) with a protein or a peptide. The resulting conjugates have the general formula:

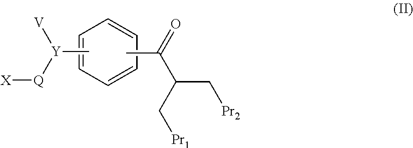

in which X, Q, Y and V have the meanings given above, and either each of Pr₁ and Pr₂ represents a separate protein or peptide molecule, or Pr₁ and Pr₂ together represent a single protein or peptide Pr bonded at two separate points to obtain the following formula (IIa):

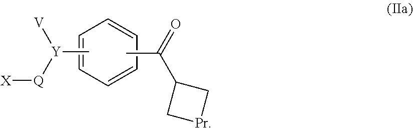

Preferably, Pr₁ and Pr₂ together represent a single protein bonded to two sulfur atoms derived from a disulfide bond in a protein or to two histidine residues present in a polyhistidine tag attached to a single protein.

In the reagent of formula (1), Z represents either —CH(CH₂L)₂ or —C(CH₂L)(═CH₂). These two groups are chemically equivalent to each other. If a reagent of formula (1) in which Z represents —CH(CH₂L)₂, i.e., a reagent of formula (Ia):

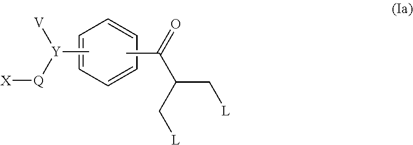

is used to react with a protein in a process according to the invention, the reaction proceeds by the loss of one leaving group L and results in the formation of a reagent of formula (Ib) in which Z represents —C(CH₂L)(═CH₂):

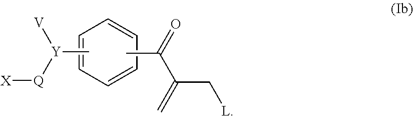

This reagent reacts with one nucleophile (for example a cysteine, histidine or lysine residue) in the protein. Subsequently, the remaining leaving group L is lost, and a reaction with a second nucleophile (either in a second molecule of a protein or in the same protein molecule as the first nucleophile) occurs to form the desired conjugate. Therefore, the process of the invention can be carried out using a compound of formula (Ia) as a starting material wherein a compound of formula (Ib) is formed in situ, or a pre-formed compound of formula (Ib) may be used as a starting material.

In another embodiment, the present invention is directed to protein conjugates of the general formula (II):

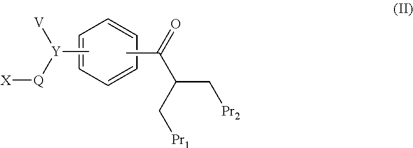

in which X, Q, Y and V have the meanings given above, and each of Pr₁ and Pr₂ independently represents a separate protein or peptide molecule, or Pr₁ and Pr₂ together represent a single protein or peptide Pr bonded at two separate points to obtain the following formula (IIa):

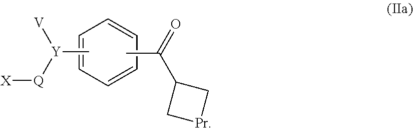

Preferably, Pr₁ and Pr₂ together represent a single protein bonded to two sulfur atoms derived from a disulfide bond in a protein or to two histidine residues present in a polyhistidine tag attached to a single protein.

In some embodiments, V may represent a homo- or co-polymer selected from the group consisting of polyalkylene glycols, polyvinylpyrrolidones, polyacrylates, polymethacrylates, polyoxazolines, polyvinylalcohols, polyacrylamides, polymethacrylamides, HPMA copolymers, polyesters, polyacetals, poly(ortho ester)s, polycarbonates, poly(imino carbonate)s, polyamides, copolymers of divinylether-maleic anhydride and styrene-maleic anhydride, polysacoharides, and polyglutamic acids. Preferably, V is discrete or non-discrete, branched or linear polyethylene.

In some embodiments, protein conjugates according to the invention have the formula:

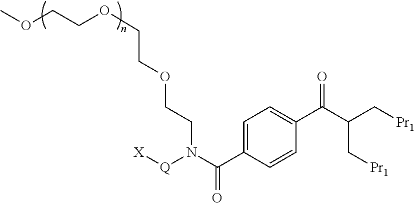

In these reagents, X is preferably a payload; and Q is a linker connecting a payload to an amide group; and n=1-5000.

In yet another embodiment, protein conjugates according to the invention have the formula:

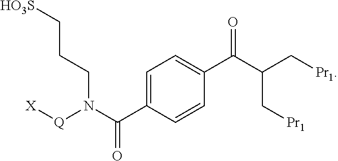

In these reagents, X is preferably a payload; and Q is a linker connecting a payload to an amide group.

In yet another embodiment, reagents according to the invention have the formula:

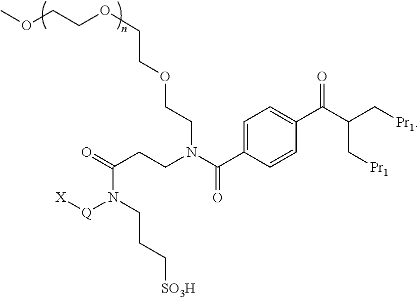

In these reagents, X is preferably a payload; Q is a linker connecting a payload to an amide group; and n=1-5000.

The conjugation reaction according to the invention may be carried out under the reaction conditions described in WO 2005/007197 and WO 2009/047500. The process may, for example, be carried out in a solvent or solvent mixture in which all reactants are soluble. For example, the protein may be allowed to react directly with the conjugation reagent in an aqueous reaction medium. This reaction medium may also be buffered, depending on the pH requirements of the nucleophile. The optimum pH for the reaction will generally be at least 4.5, typically between about 5.0 and about 8.5, preferably about 6.0 to 7.5. The optimal reaction conditions will of course depend upon the specific reactants employed.

Reaction temperatures between 4-37° C. are generally suitable when using an aqueous reaction medium. Reactions conducted in organic media (for example THF, ethyl acetate, acetone) are typically conducted at temperatures up to ambient. The reaction temperature is preferably between 4-45° C. and more preferably at ambient temperature.

Where bonding to the protein is via two sulfur atoms derived from a disulfide bond in the protein, the process may be carried out by reducing the disulfide bond in situ and then reacting the reduced product with the reagent of the formula (1). Preferably, the disulfide bond is reduced, and any excess reducing agent is removed, for example, by buffer exchange, before the conjugation reagent is introduced. The disulfide can be reduced, for example, with dithiothreitol, mercaptoethanol, or tris-carboxyethylphosphine using conventional methods.

The protein can be effectively conjugated using a moderate excess of conjugation reagent I. The excess reagent can easily be removed, for example, by ion exchange chromatography, during subsequent purification of the conjugate.

Compounds of the general formula (1) in which Z represents —CH(CH₂L)₂ or —C(CH₂L)(═CH₂) may be prepared by reacting a compound of the general formula (III)

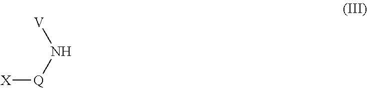

with a compound of the general formula (IV) or (IVa)

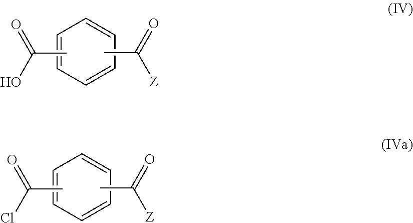

to form an amide bond. It is well known in the art that the CO₂H group can be reacted with secondary amines to form the amide group in the presence of suitable activators (e.g., EDC or HATU) to facilitate the reaction. Alternatively, carboxylic acid can be converted into an activated ester, an acyl chloride, or an anhydride and reacted with secondary amines in the absence of activators.

The compounds of the general formula (IV) in which Z represents —CH(CH₂L)₂ or —C(CH₂L)(═CH₂) may be prepared as described in Rosario et al., Bioconjugate Chemistry 1 (1990) 51-59 and Liberatore et al., Bioconjugate Chemistry 1 (1990) 36-50.

Similar to the process described above, another embodiment of the present invention is directed to a process for the preparation of a protein conjugate of the general formula (3):

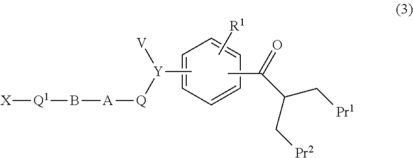

wherein:
X represents a payload;
Q and Q¹ independently represent a cleavable or non-cleavable linking group;
Y represents an amide group;
V represents a moiety that improves cross-linking and/or labeling efficiency or is absent;
R¹ represents H, alkyl, —CN, —NO₂, —CO₂R, —COH, —CH₂OH, —COR², —OR², —OCOR², —OCO₂R², —SR², —SO, —SO₂R², —NHCOR², —NR²COR², —NHCO₂R², —NO, —NHOH, —NR²OH, —C═N—NH—COR², halogen, —C═CR², —C═CR²₂, or —C═CHR, in which each R² independently represents a hydrogen atom or alkyl, aryl, or alkyl-aryl group;
both Pr¹ or Pr² together represent a single protein or peptide bonded at two separate points via two thiol groups generated by the reduction of a disulfide bridge in the protein;

A-B is a pair of orthogonally reactive moieties that can react with each other without activation and are stable under commonly applied protein labeling conditions;
said method comprising the steps of:
(a) reducing a disulfide bridge in the protein; and
(b) reacting the reduced protein with a compound of general formula (2) to form an activated protein of general formula (4):

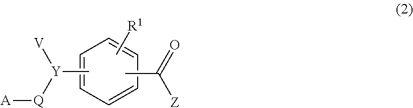

wherein Z represents either —CH—(CH₂L)₂ or —C(CH₂L)(═CH₂), L independently represents a leaving group;

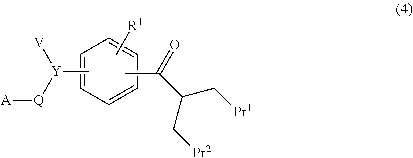

(c) reacting said activated protein with a compound of general formula (5):

X-Q¹-B (5).

In the method, L may represent —SR³, —SO₂R³, —OSO₂R³, —N⁺R³₃, —N⁺HR³₂, —N⁺H₂R³, halogen, or —O◯, in which R³ represents a hydrogen atom or an alkyl, aryl, or alkyl-aryl, and ◯ represents a substituted aryl group containing at least one electron withdrawing substituent.

V may represent H. V may represent —(CH₂)ₙSO₃X, in which n=1-6 and X represents H or counterion. V may represent a hydrophilic polymer.

A may be selected from the group consisting of orthogonal reactive pairs that undergo Staudinger ligation, strain-promoted Huisgen 1,3-cycloaddition, Inverse Demand Diels-Alder cycloaddition, and hydrazone or oxime bond forming reactions. A may represent a 1,3-dipole group for example, azide, nitrile oxide, nitrone, azoxy group, and acyl diazo group. A may represent a substituted or unsubstituted cyclooctyne that undergoes a 1,3-cycloadditon reaction with a 1,3 dipole group. A may represent a diene that undergoes an Inverse electron-demand Diels-Alder reaction, for example a substituted or unsubstituted tetrazine. A may represent a dienophile that undergoes an Inverse electron-demand Diels-Alder reaction, for example a substituted or unsubstituted trans-cyclooctene.

X may represent a drug, a diagnostic moiety, or a chelating agent. X may represent a biopolymer such as protein or a synthetic polymer such as PEG.

X, Q, Y, V, Z, A, B, R, Pr¹, and Pr² may also be defined the same as discussed above for the first process or as in formula (1). Q¹ of this process may be independently defined the same as Q as discussed above for the first process or as in formula (1).

It has been discovered that the conjugation of moieties (e.g., chemical entities) to antibodies using bis-sulfone conjugation reagents of general formula (1) in combination with low-to-moderately hydrophobic payloads, such as biotin, trans-cyclooctene, dibenzylcoclooctyne, tetrazine, azide, TAMRA, or fluorescein, results in very moderate coupling and cross-linking. At the same time, the conjugation of moieties (e.g., chemical entities) to antibodies using bis-sulfone conjugation reagents of general formula (1) in combination with highly hydrophobic compounds, such as MMAF, provides, at best, only partial conjugation. The conjugation appears to be dependent, among other factors, on the nature of the payload. While smaller and low-to-moderately hydrophobic substrates, such as biotin, can be coupled with high cross-linking using a known single-step method, some substrates, such as hydrophobic toxin substrates, are poorly coupled, leading to low degree of cross-linking and to heterogeneous mixtures of antibodies or not coupled at all. Coupling reaction parameters were optimized but could not resolve the problems of low levels of coupling and thus product homogeneity.

Another shortfall of the one-step conjugation approach using bis-sulfone conjugation reagents of general formula (1) is that it is poorly applicable to the preparation of protein-protein conjugates. Beside low cross-linking efficiency, usually a large excess of one protein should be used, which might be impractical due to the relatively high cost of proteins and the need for the removal of the protein-in-excess after the conjugation step.

One embodiment of this invention provides a method of efficiently conjugating bis-sulfone conjugation reagents of general formula (1) in combination with highly hydrophobic payloads, such as MMAF, duocarmycins, maytansanoids, alkylating agents, taxanes, or auristatins, to large molecules, such as proteins or large synthetic polymers.

In yet another embodiment, the present invention relates to a method for conjugating a moiety of interest to an antibody, comprising the steps of:

a) reducing an antibody or fragment having at least one disulfide bond in situ and then reacting the reduced product with the reagent of the formula (V), where B represents a bioorthogonal reactive moiety, and Z, Y, V, and Q have the meanings given above, to form an activated antibody of the general formula (VI);

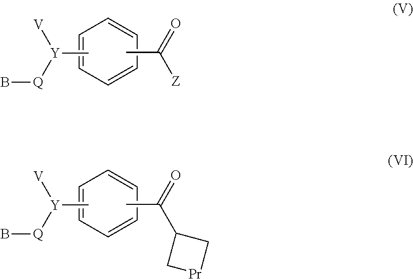

b) reacting said activated antibody of the general formula (VI) with a linker of the general formula (VII) in which P represents a payload of interest, Q₁ is a linker, and A is a complimentary bioorthogonal group, to form an antibody conjugate of the general formula (IX).

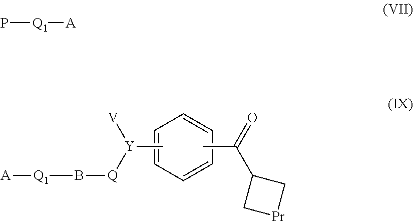

Preferably, the disulfide bond is reduced, and any excess reducing agent is removed, for example, by buffer exchange, before the conjugation reagent is introduced. The disulfide can be reduced, for example, with dithiothreitol, mercaptoethanol, or tris-carboxyethylphosphine using conventional methods. It is also preferable to remove an excess of the reagent of the formula (V). A two-step approach for protein conjugation takes advantage of bioorthogonal ligation chemistry. The term bioorthogonal chemistry refers to any chemical reaction that can occur in the presence of rich functionalities of biological media without interfering with native biochemical processes. In this strategy, a small linker containing bioorthogonal functionality is introduced onto a protein. The payload, which is equipped with a complementary functional group reactive toward the linker, is then reacted with the protein-linker conjugate to yield the desired conjugate.

The first step of above described strategy takes advantage of the selectivity of the bis-sulfone functional groups to introduce a new bioorthogonal reactive group that then provides superior reaction kinetics for a second, more complex conjugation reaction. Using this strategy, a large excess of the initial reagent can be used to introduce a reactive handle into a protein, followed by a second reaction using near stoichiometric quantities of the payload of interest.

It is not uncommon that changing the nature of a labeling reagent requires fine tuning of the conjugation conditions (e.g., temperature, concentrations, reaction time, pH of media, etc.). The protein labeling optimization might be a tedious process. It is highly desirable to be able to attach different payloads without having to optimize the protein labeling procedure. The two-step conjugation method of the invention allows for the conjugation of different payloads without changing the protein labeling procedure, simply by changing the payload on a linker containing the bioorthogonal group. In general, bioorthogonal reactions are independent of the nature of the payload attached to it and the pH of the media. In addition, the use of the two-step process provides conjugates of the same degree of cross-linking and conjugation.

In preferred embodiments, the reactive bioorthogonal pair A-B is chosen to undergo Staudinger ligation, Huisgen 1,3-cycloaddition (click reaction), Cu-free click reaction, Inverse Demand Diels-Alder cycloaddition, or hydrazone or oxime bond forming reactions.

Exemplary payloads might be a therapeutic moiety, a diagnostic moiety, or any other moiety for a desired function that is otherwise difficult to conjugate with the reagent of general formula (1) in one step.

In some embodiments, reagents according to the invention have the formula:

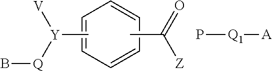

In these reagents, A-B is the reactive bioorthogonal pair capable of undergoing Huisgen 1,3-cycloaddition in the absence of a catalyst (Cu-Free click reaction), and Q, Y, and V have the meanings given above. In addition, in these reagents, each L is preferably a tosyl group. In preferred embodiments, reagents according to the invention capable of undergoing Huisgen 1,3-cycloaddition in the absence of a catalyst have the formula:

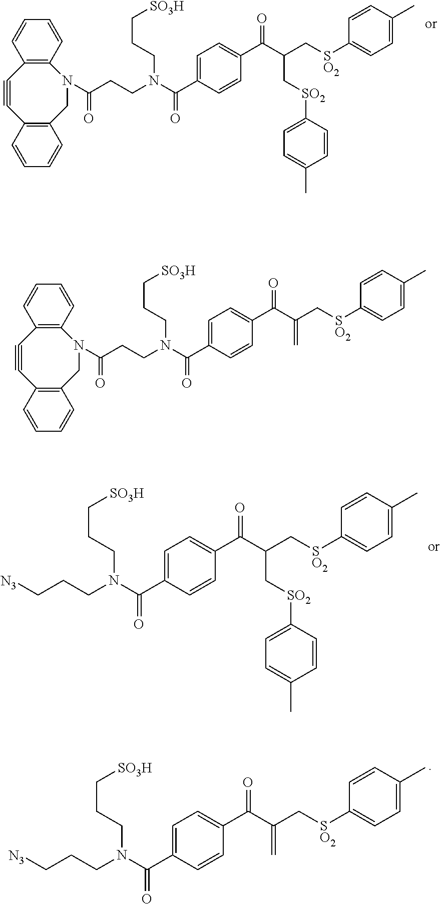 [SMILES: Cc1ccc(S(=O)(=O)CC(CS(=O)(=O)c2ccc(C)cc2)C(=O)c2ccc(C(=O)N(CCCS(=O)(=O)O)CCC(=O)N3Cc4ccccc4C#Cc4ccccc43)cc2)cc1 ; C=C(CS(=O)(=O)c1ccc(C)cc1)C(=O)c1ccc(C(=O)N(CCCS(=O)(=O)O)CCC(=O)N2Cc3ccccc3C#Cc3ccccc32)cc1 ; Cc1ccc(S(=O)(=O)CC(CS(=O)(=O)c2ccc(C)cc2)C(=O)c2ccc(C(=O)N(CCCN=[N+]=[N-])CCCS(=O)(=O)O)cc2)cc1 ; C=C(CS(=O)(=O)c1ccc(C)cc1)C(=O)c1ccc(C(=O)N(CCCN=[N+]=[N-])CCCS(=O)(=O)O)cc1]

A variety of compounds having at least one 1,3-dipole group (having a three-atom pi-electron system containing 4 electrons delocalized over the three atoms) can be used to react with the alkynes disclosed herein. Exemplary 1,3-dipole groups include, but are not limited to, azides, nitrile oxides, nitrones, azoxy groups, and acyl diazo groups.

Cycloalkynes, including specific compounds, are described, for example, in U.S. Pat. No. 7,807,619, the disclosure of which is incorporated herein by reference.

In some embodiments, reagents according to the invention have the formula:

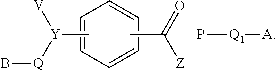

In these reagents, A-B is the reactive bioorthogonal pair capable of undergoing Inverse Demand Diels-Alder cycloaddition, and Q, Y, and V have the meanings given above. In addition, in these reagents, each L is preferably a tosyl group.

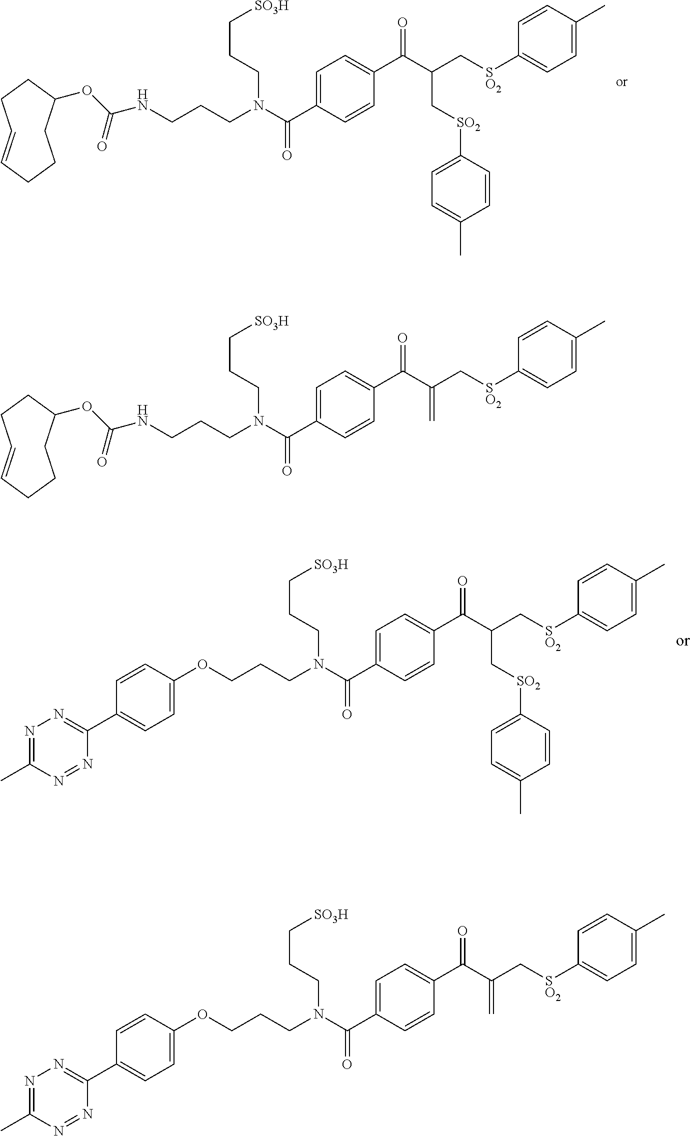

In some embodiments, the present invention relates to reagents and a process for preparing protein-protein and protein-polymer conjugates. One specific example is the conjugation of an HRP enzyme to an antibody or antibody fragment using novel reagents and methods as disclosed in this invention. An antibody or fragment can be activated with bis-sulfone reagents that contain a bioorthogonal relative group, and separately, HPR enzyme is activated with a complementary bioorthogonal reactive group. Then, both activated proteins are mixed to form the desired conjugate. In preferred embodiments, the reactive bioorthogonal pair is trans-cycloocteen and tetrazine, a pair of reagents that undergoes Inverse Demand Diels-Alder cycloaddition.

In yet another embodiment, an antibody or fragment can be activated with reagents of bis-sulfone reagent that contains a bioorthogonal relative group, and separately, synthetic polymer (e.g., mPEG 20 kDa) is activated with a complementary bioorthogonal reactive group. Then, both activated proteins are mixed to form the desired conjugate. In preferred embodiments, the reactive bioorthogonal pair is trans-cycloocteen and tetrazine, a pair of reagents that undergoes Inverse Demand Diels-Alder cycloaddition.

The invention further provides a pharmaceutical composition comprising a conjugate according to the invention together with a pharmaceutically acceptable carrier, and optionally also contains a further active ingredient in addition to the conjugate according to the invention; a conjugate according to the invention for use in therapy; the use of a conjugate according to the invention in a process for the manufacture of a medicament; and a method for treating a patient, which comprises administering a pharmaceutically-effective amount of a conjugate or a pharmaceutical composition according to the invention to a patient.

The conjugation reagents and methods of the present invention have been found to be extremely useful, being capable of highly efficient site-specific conjugation of proteins with small molecules, proteins, and synthetic polymers.

While the invention has been described with references to preferred embodiments, those skilled in the art will understand various changes may be made and equivalents may be substituted for elements thereof without departing from the scope of the invention. In addition, many modifications may be made to adapt a particular situation or materials to the teaching of the invention without departing from the essential scope thereof. Therefore, it is intended that the invention not to be limited to the particular embodiments disclosed but that the invention will include all embodiments falling within the scope of the appended claims. In this application, all units are in the metric system, and all amounts and percentages are by weight, unless otherwise expressly indicated. Also, all citations referred to herein are expressly incorporated herein by reference.

The following examples are offered to illustrate various embodiments of the invention but should not be viewed as limiting the scope of the invention.

### EXAMPLES

### Example 1

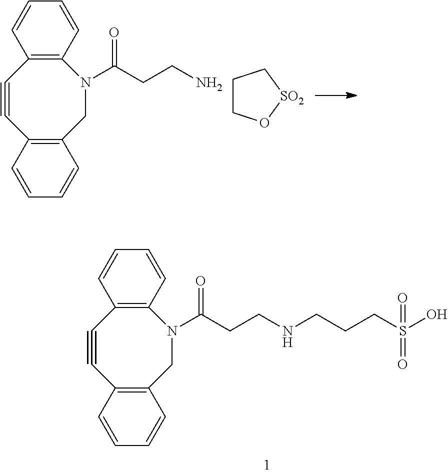

A solution of DBCO-Amine (4 g, 14.48 mmol) and 1,2-oxathiolane 2,2-dioxide (1.591 g, 13.03 mmol) in DCM (20 mL) was stirred overnight at room temperature. The white precipitate was filtered, washed with a small amount of THF-Et₂O, and dried on an oil pump to provide 3.53 g (8.86 mmol, 61%) of compound 1 that was used without any further purification.

### Example 2

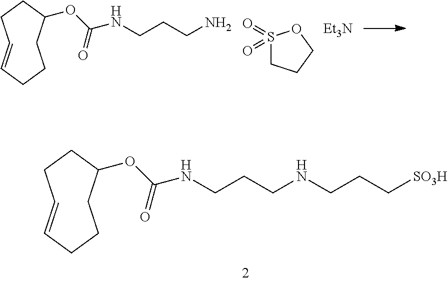

A solution of TCO-Amine, HCl salt (0.45 g, 1.80 mmol), 1,2-oxathiolane 2,2-dioxide (0.24 g, 1.99 mmol), and Et₃N (0.20 g, 1.99 mmol) in DCM (10 mL) was stirred overnight at room temperature. The reaction mixture was concentrated and chromatographed on silica gel to provide 0.26 g (0.75 mmol. 37% yield) of compound 2 as a waxy solid.

### Example 3

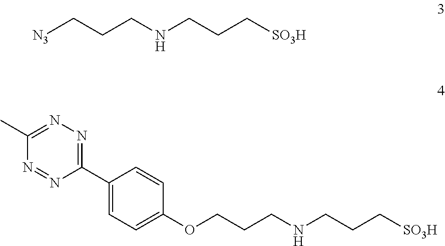 [SMILES: [N-]=[N+]=NCCCNCCCS(=O)(=O)O ; Cc1nnc(-c2ccc(OCCCNCCCS(=O)(=O)O)cc2)nn1]

Compounds 3 and 4 were prepared according to Examples 1 or 2 using azido-propylamine or tetrazine-amine, HCl salt, both of which are commercially available.

### Example 4

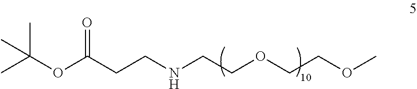

Tert-butyl acrylate (6.87 g, 53.6 mmol) was added to a solution of mPEG11-amine (10 g 17.87 mmol) (Quanta Biodesign, Limited) in ethanol (100 mL) at room temperature, and the reaction mixture was stirred overnight at room temperature. The reaction mixture was concentrated and chromatographed on silica gel to provide 8.25 g (11.99 mmol, 67% yield) of compound 5 as slightly yellow oil.

### Example 5

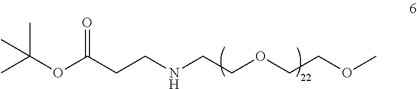

Compound 6 was prepared according to Example 4 using mPEG23-amine (Quanta Biodesign, Limited).

### Example 6

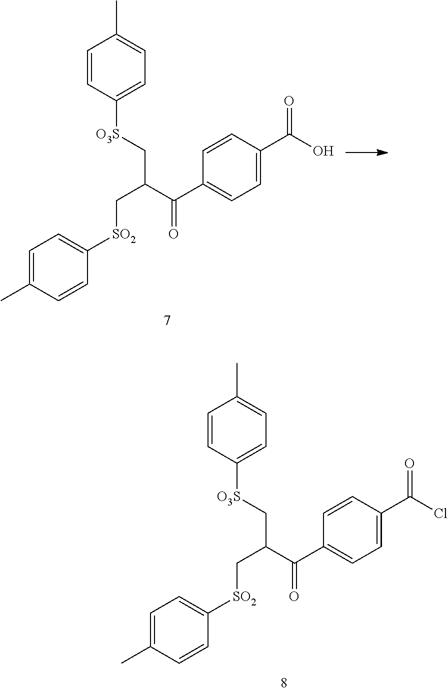

Compound 7 (5 g) (prepared according to Rosario et al., Bioconjugate Chemistry 1 (1990) 51-59, Liberatore et al., Bioconjugate Chemistry 1 (1990) 36-50) was dissolved in 50 mL of dichloromethane at room temperature. Oxalyl chloride (5 mL) was added, followed by a drop of DMF. The reaction mixture was stirred for 5 hours at room temperature, concentrated, and residual white solid was dried on an oil pump for 2 hours. The crude compound 8 was used without any further purification.

### Example 8

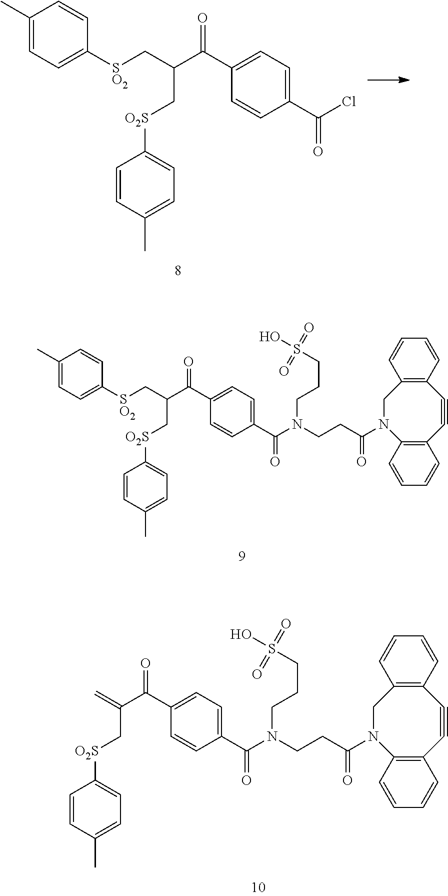 [SMILES: Cc1ccc(S(=O)(=O)CC(CS(=O)(=O)c2ccc(C)cc2)C(=O)c2ccc(C(=O)Cl)cc2)cc1 ; Cc1ccc(S(=O)(=O)CC(CS(=O)(=O)c2ccc(C)cc2)C(=O)c2ccc(C(=O)N(CCCS(=O)(=O)O)CCC(=O)N3Cc4ccccc4C#Cc4ccccc43)cc2)cc1 ; C=C(CS(=O)(=O)c1ccc(C)cc1)C(=O)c1ccc(C(=O)N(CCCS(=O)(=O)O)CCC(=O)N2Cc3ccccc3C#Cc3ccccc32)cc1]

A suspension of compound 8 (160 mg, 0.38 mmol) in DCM (5 mL) was cooled to ca. 4° C. in an ice-water bath. A solution of compound 1 (134 mg, 0.336 mmol) and Et₃N (65 mg, 0.64 mmol) in DCM (ca. 1 mL) was added in one portion, and the resulting mixture was stirred for ca. 3 hours at 0° C. According to TLC analysis (SiO₂, 10:1 DCM:MeOH), the reaction was complete. The crude reaction was directly loaded on a silica gel column and chromatographed on silica gel (10:1 DCM:MeOH) to provide 0.23 g (0.261 mmol, 85% yield) of a white solid that mainly consisted of compound 9. Compounds can be easily separated by prep-HPLC purification.

### Example 9

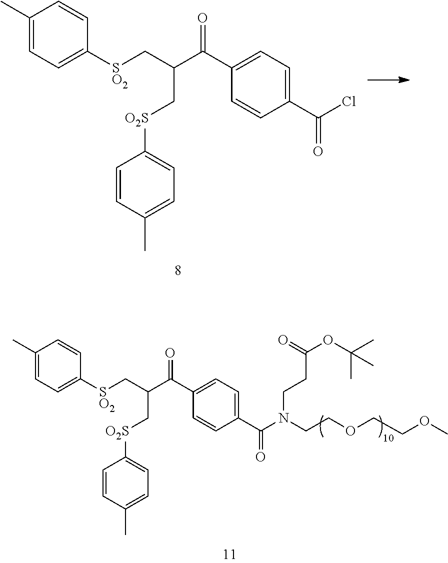

A suspension of compound 8 (520 mg, 1.002 mmol) in DCM (10 mL) was cooled to ca. 4° C. in an ice-water bath. A solution of compound 5 (647 mg, 1.005 mmol) and Et₃N (116 mg, 1.148 mmol) in DCM (ca. 3 mL) was added in one portion, and the resulting mixture was stirred for ca. 3 hours at 0° C. According to TLC analysis (SiO₂, 10:1 DCM:MeOH), the reaction was complete. The crude reaction was directly loaded on a silica gel column and chromatographed on silica gel (EtOAc:MeOH 50:1 to 50:6) to provide 1.06 g of colorless oil that according to HPLC and LC/MS was a mixture of bis-tosyl:mono-tosyl 9:1.

### Example 10

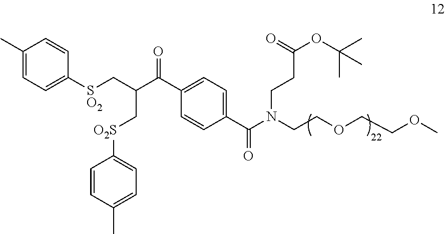

Compound 12 was prepared according to Example 9.

### Example 11

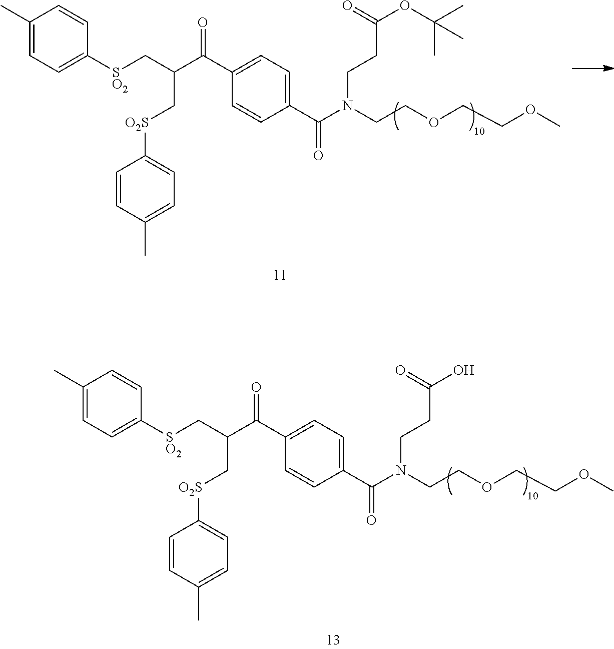

A solution of compound 11 (1.06 g) in DCM 10 mL was cooled to ca. 4° C. in an ice-water bath, TFA (3 mL) was added dropwise, and the reaction mixture was stirred for ca. 5 hours at 4° C. According to TLC analysis, all tetr-butyl ester 11 was converted into an acid 13. The reaction mixture was concentrated under reduced pressure and co-evaporated with toluene (2×10 mL) to provide a slightly yellow oil that was used without any further purification.

### Example 12

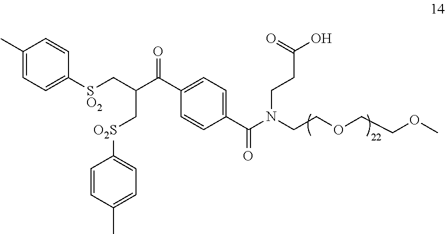

Compound 14 was prepared according to Example 11.

### Example 13

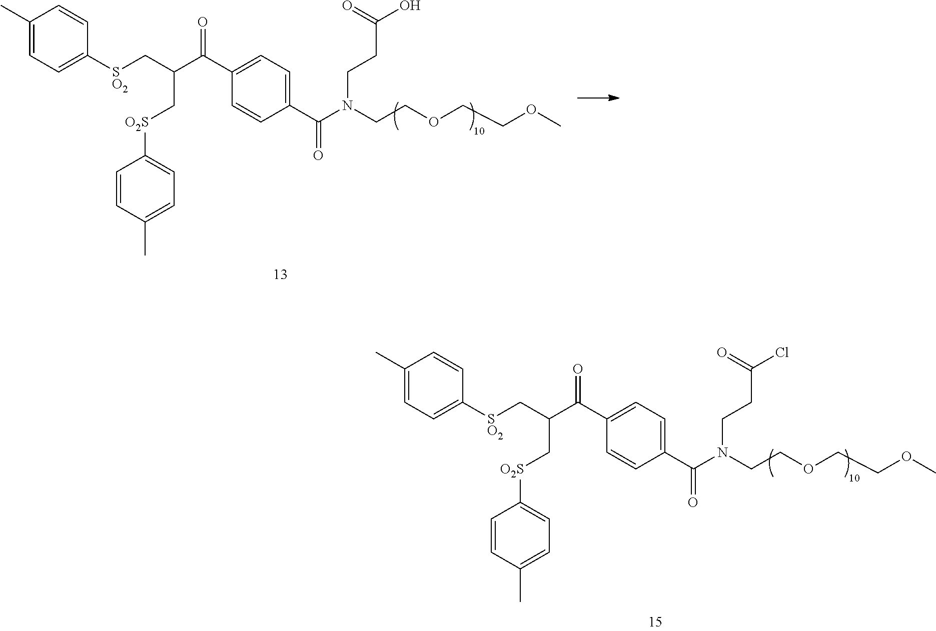

To a cooled solution of compound 13 (1.03 g) in DCM 10 mL (ca. 4° C. in an ice-water bath) was added oxalyl chloride (580 mg, 4.57 mmol) followed by a drop of DMF, and the reaction mixture was stirred for ca. 2 hours at room temperature. The reaction mixture was concentrated under reduced pressure and co-evaporated with toluene (2×10 mL) to provide a slightly yellow oil that was used without any further purification.

### Example 14

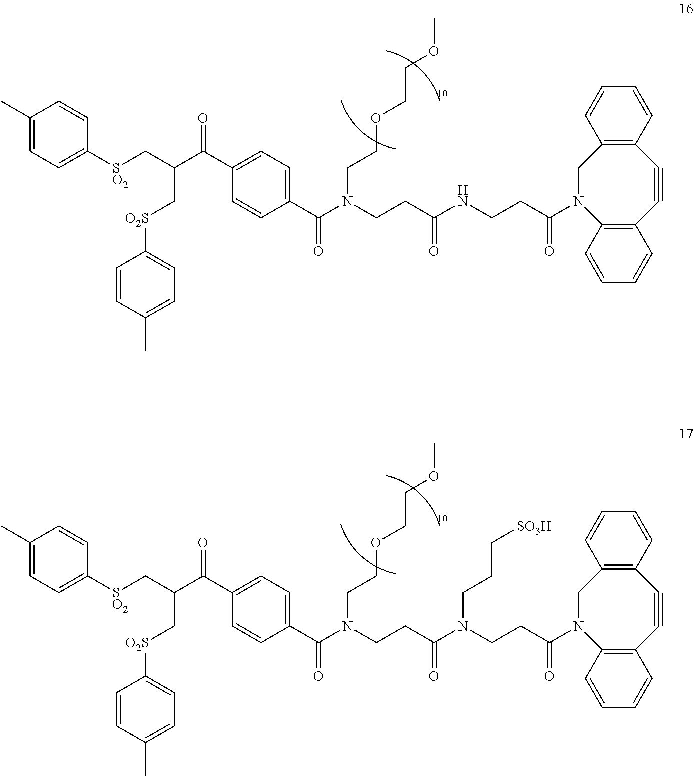

Compounds 16 and 17 were prepared according to Example 8.

### Example 15

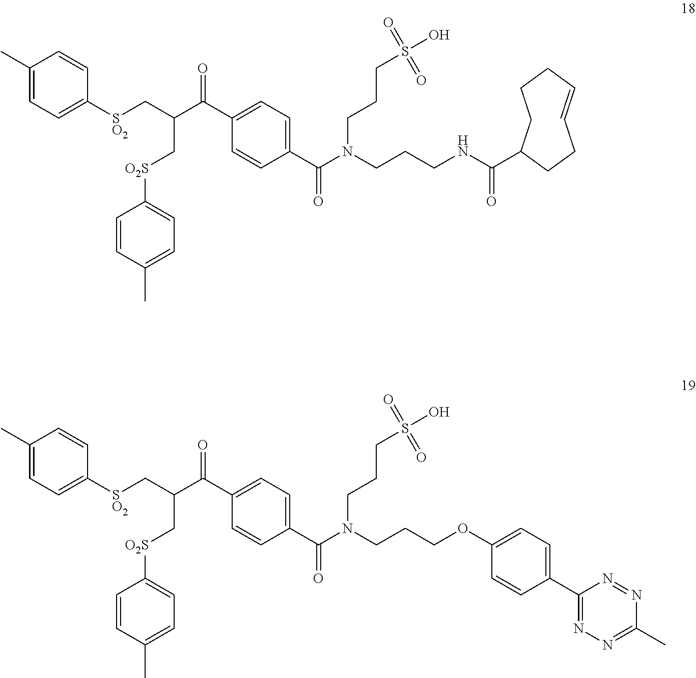

Compounds 18 and 19 were prepared according to Example 8.

### Example 16

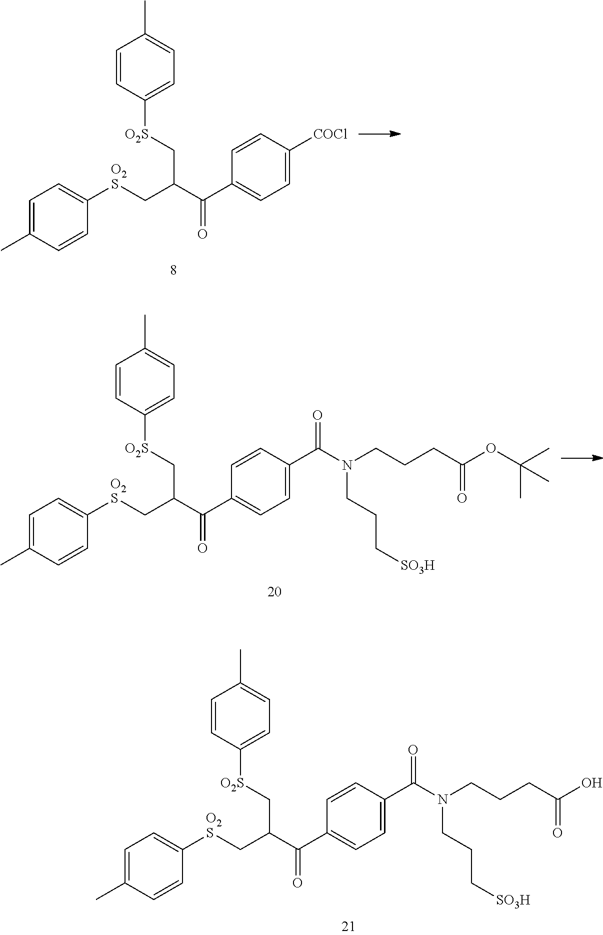

A solution of Et₃N (0.585 ml, 4.05 mmol) and 3-((4-(tert-butoxy)-4-oxobutyl)amino)propane-1-sulfonic acid (0.542 g, 1.927 mmol) was cooled to −78° C. on a dry ice-acetone bath. A solution of compound 8 (1 g, 1.927 mmol) in DCM (ca. 3 mL) was added, and the reaction mixture was stirred for 60 min at −78° C. and warmed to room temperature. The reaction was quenched by addition of 1 mL of acetic acid, absorbed on silica gel, and purified on silica gel to provide 1.05 g of compound 20 as a white solid. The freshly purified t-Bu ester (1.05 g) was dissolved in 4 M HCl in dioxane and stirred for 3 hours at room temperature. The reaction mixture was concentrated and dried on an oil pump overnight. The crude compound 21 was used without any further purification.

### Example 17

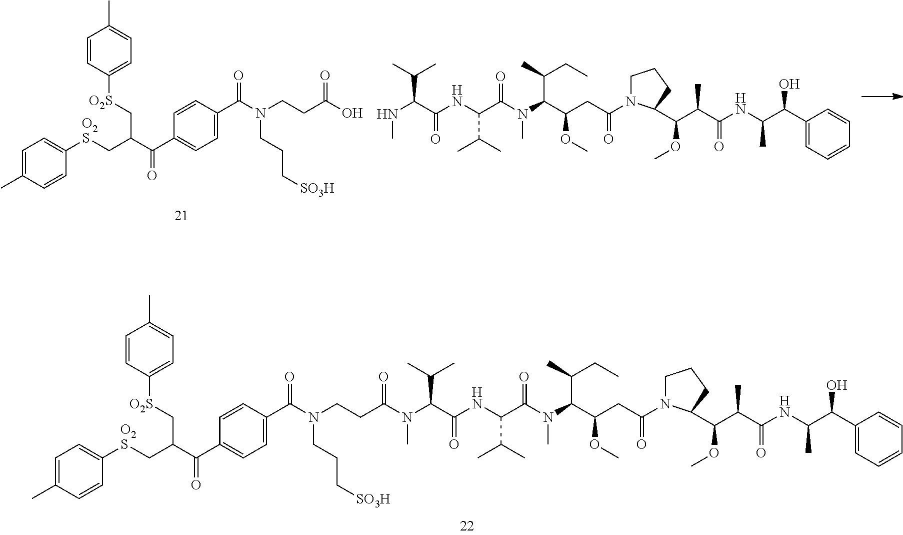 [SMILES: Cc1ccc(S(=O)(=O)CC(CS(=O)(=O)c2ccc(C)cc2)C(=O)c2ccc(C(=O)N(CCCS(=O)(=O)O)CCC(=O)O)cc2)cc1 ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)CCN(CCCS(=O)(=O)O)C(=O)c1ccc(C(=O)C(CS(=O)(=O)c2ccc(C)cc2)CS(=O)(=O)c2ccc(C)cc2)cc1)C(C)C]

HATU (0.041 g, 0.108 mmol) was added to a mixture of compound 21 (0.064 g, 0.094 mmol), MMEA (0.05 g, 0.070 mmol), and DIEA (0.029 ml, 0.174 mmol) at room temperature, and the reaction mixture was stirred for ca. 30 min. Upon completion (LC/MS), the reaction was concentrated and purified on preparative HPLC (C-18, Water:MeOH 15% to 75% over 30 min).

### Example 18

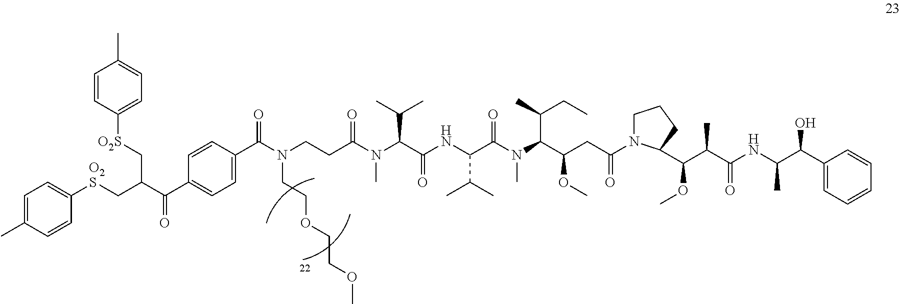

Compound 23 was prepared according to Example 17.

### Example 19

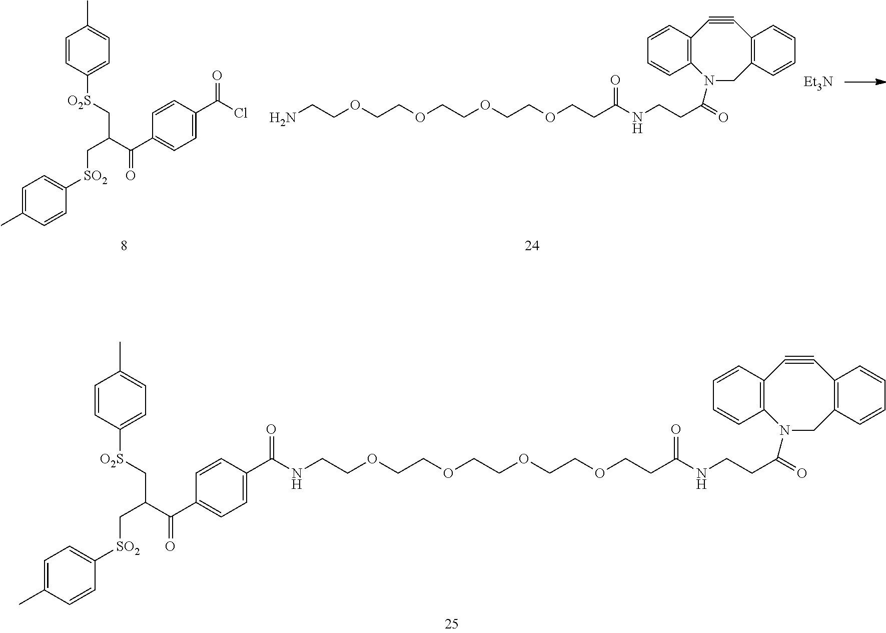 [SMILES: "Et3N" ; Cc1ccc(S(=O)(=O)CC(CS(=O)(=O)c2ccc(C)cc2)C(=O)c2ccc(C(=O)Cl)cc2)cc1 ; NCCOCCOCCOCCOCCC(=O)NCCC(=O)N1Cc2ccccc2C#Cc2ccccc21 ; Cc1ccc(S(=O)(=O)CC(CS(=O)(=O)c2ccc(C)cc2)C(=O)c2ccc(C(=O)NCCOCCOCCOCCOCCC(=O)NCCC(=O)N3Cc4ccccc4C#Cc4ccccc43)cc2)cc1]

A solution of Et₃N (0.202 g, 2.00 mmol) and DBCO-PEG4-Amine (24) (0.76 g, 1.46 mmol) was cooled to −78° C. on a dry ice-acetone bath. A solution of compound 8 (0.75 g, 1.45 mmol) in DCM (ca. 3 mL) was added, and the reaction mixture was stirred for 60 min at −78° C. and warmed to room temperature. The reaction was absorbed on silica gel and purified on silica gel to provide 1.14 g of compound 25 as a white amorphous solid.

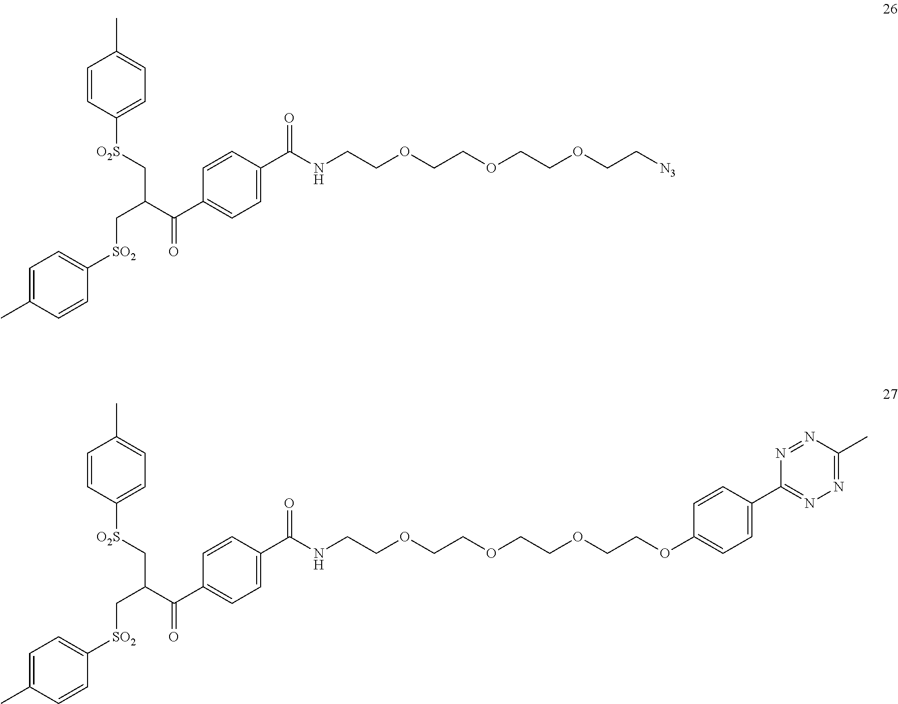

Compounds 26 and 27 were prepared according to Example 19.

### General Protocol for Conjugation to IgG Using Compound 9, 16, 17, 18, or 19.

IgG (5 mg/mL) in 20 mM sodium phosphate pH 8.0, 150 mM NaCl, 20 mM EDTA was treated with tris(2-carboxyethyl)phosphine (TCEP, 5 mM) at room temperature for 30 min. The reductant was then removed by buffer exchange into fresh pH 6.5 phosphate buffer by gel filtration (7 kDa cutoff, Zeba column, Pierce Biotechnology). Determination of free thiols using 5,5′-dithiobis(2-nitrobenzoic acid) showed that there were approximately four SH groups per IgG. A solution of compound 9, 16, 17, 18, or 19 (4 equiv per reduced thiol bridge) in water was added, and the resulting mixture incubated at 37° C. for 3 hours. Unreacted reagent was removed by gel filtration.

Determination of degree of labeling (DOL) was performed by reacting an aliquot of IgG conjugate with 20 molar equivalents of Cy5-azide, Cy5-TCO, and Cy-Tetrazine (all are available from Click Chemistry Tools, Scottsdale, Ariz., USA) overnight, followed by removing any excess of unreacted Cy5 dyes. DOL for all compounds was determined to be within the range of 2.8-3.2. It was possible to tailor reaction conditions to obtain a lower or higher DOL.

### General Protocol for Conjugation to FAB Using Compound 9, 16, 17, 18, or 19.

FAB (2 mg/mL, Jackson Immuno Research Laboratories, Inc, cat#009-000-007) in 20 mM sodium phosphate pH 8.0, 150 mM NaCl, 20 mM EDTA was treated with tris(2-carboxyethyl)phosphine (TCEP, 20 mol equiv) at room temperature for 30 min. The reductant was then removed by buffer exchange into fresh pH 6.5 phosphate buffer by gel filtration (7 kDa cutoff, Zeba column, Pierce Biotechnology). Compound 9, 16, 17, 18, or 19 (3 equiv per reduced thiol bridge) in water was added, and the resulting mixture incubated at 37° C. overnight. Unreacted reagent was removed by gel filtration.

Determination of degree of labeling (DOL) was performed by reacting an aliquot of IgG conjugate with 20 molar equivalents of Cy5-azide, Cy5-TCO, and Cy-Tetrazine (all are available from Click Chemistry Tools, Scottsdale, Ariz., USA), and the crosslinking efficiency was determined by reducing SDS-PAGE.

### General Protocol for Conjugation of Pre-Activated IgG or FAB to Small Molecule Payloads.

To a solution of FAB (1-1.5 mg/mL) or IgG (1.5-4 mg/mL) in 20 mM sodium phosphate pH 7.5, 150 mM NaCl, was added a solution of an azide-payload (2-6 eq per DBCO group) in DMSO, and the resulting mixture incubated at 37° C. for 12 hours. Unreacted reagent was removed by gel filtration.

### Example 20

### Conjugation of IgG Pre-Activated with Compound 8 to Doxorubicin-PEG4-Azide

Doxorubicin hydrochloride (0.2 g, 0.346 mmol) was added to a solution of Azide-PEG4-NHS Ester (0.155 g, 0.398 mmol) in DMF (Volume: 4 ml) followed by triethylamine (0.070 g, 0.692 mmol) at room temperature, and the reaction mixture was stirred overnight at room temperature. According to LC/MS, all Doxorubicin was consumed, and one major product was formed. The reaction mixture was concentrated under reduced pressure, and the product was purified on silica gel (DCM:MeOH 0% to 20% over 15 min) to provide Doxorubicin-PEG4-azide (0.19 g, 0.233 mmol, 67.2% yield) as a dark orange waxy solid.

A stock solution of Doxorubicin-PEG4-azide (3 fold excess per DBCO group) in DMSO was added to a solution of IgG (2.3 mg/mL, 3.1 DBCO groups per IgG) in 20 mM sodium phosphate pH 7.5, 150 mM NaCl, and the resulting mixture incubated at 37° C. overnight. Unreacted reagent was removed by gel filtration.

### Example 21

### Conjugation of FAB Pre-Activated with Compound 9 to Doxorubicin-PEG4-Azide

A stock solution of Doxorubicin-PEG4-azide (5 fold excess per DBCO group) in DMSO was added to a solution of FAB (1.6 mg/mL) in 20 mM sodium phosphate pH 7.5, 150 mM NaCl, and the resulting mixture incubated at 37° C. overnight. Unreacted reagent was removed by gel filtration.

In a similar fashion, pre-activated IgG and FAB were conjugated to Biotin-PEG3-Azide, TAMRA-azide, Cy5.5-azide and DOTA-azide (Macrocyclics, Dallas, Tx).

### Example 22

### General Protocol for Conjugation of IgG or FAB Pre-Activated with Compound 18 to Large Molecule Payloads

To a solution of FAB (1-1.5 mg/mL) or IgG (1.5-4 mg/mL) in 20 mM sodium phosphate pH 7.5, 150 mM NaCl, was added a solution of an tetrazine-payload (1.2-3 eq per TCO group) in water, and the resulting mixture incubated at 37° C. for 6-12 hours.

### Example 23

A stock solution of HRP-tetrazine (10 mg/mL in PBS buffer, pH 7.5, 1.5 fold excess per TCO group) was added to a solution of TCO pre-activated IgG (3.1 mg/mL, 2.8 TCO groups per IgG) in 20 mM sodium phosphate pH 7.5, 150 mM NaCl, and the resulting mixture incubated at 37° C. overnight. The formation of conjugates was confirmed by SDS-PAGE.

### Example 24

A stock solution of Tetrazine-mPEG 20 kDa (25 mg/mL in PBS buffer, pH 7.5, 1.5 fold excess per TCO group) was added to a solution of IgG (3.1 mg/mL, 2.8 TCO groups per IgG) in 20 mM sodium phosphate pH 7.5, 150 mM NaCl, and the resulting mixture incubated at 37° C. overnight. The formation of conjugates was confirmed by SDS-PAGE.

### Example 25

A stock solution of HRP-tetrazine (10 mg/mL in PBS buffer, pH 7.5, 1.5 fold excess per TCO group) was added to a solution of TCO pre-activated FAB (1.1 mg/mL) in 20 mM sodium phosphate pH 7.5, 150 mM NaCl, and the resulting mixture incubated at 37° C. overnight. The formation of conjugates was confirmed by SDS-PAGE.

### Example 26

A stock solution of Tetrazine-mPEG 20 kDa (25 mg/mL in PBS buffer, pH 7.5, 1.5 fold excess per TCO group) was added to a solution of TCO pre-activated FAB (1.1 mg/mL) in 20 mM sodium phosphate pH 7.5, 150 mM NaCl, and the resulting mixture incubated at 37° C. overnight. The formation of conjugates was confirmed by SDS-PAGE.

### Example 27

IgG (5 mg/mL) in 20 mM sodium phosphate pH 8.0, 150 mM NaCl, 20 mM EDTA was treated with tris(2-carboxyethyl)phosphine (TCEP, 5 mM) at room temperature for 30 min. The reductant was then removed by buffer exchange into fresh pH 6.5 phosphate buffer by gel filtration (7 kDa cutoff, Zeba column, Pierce Biotechnology). A stock solution of compound 22 or 23 (4 equiv per reduced thiol bridge) in DMSO was added, and the resulting mixture incubated at 37° C. for 3 hours. Unreacted reagent was removed by gel filtration.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A compound of the general formula (1): 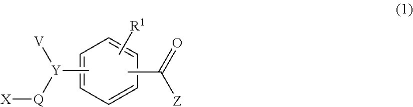
wherein
X represents a payload;
Q represents a cleavable or non-cleavable linking group;
Y represents an amide group;
V represents a moiety that improves cross-linking and/or labeling efficiency;
Z represents either —CH—(CH₂L)₂ or —C(CH₂L)(═CH₂), L independently represents a leaving group, and
R¹ represents H, alkyl, —CN, —NO₂, CO₂R, —COH, CH₂OH, COR², —OR², —OCOR², —OCO₂R², —SR², SO, —SO₂R², —NHCOR², —NR²COR², NHCO₂R², —NO, —NHOH, —NR²OH, —C═N—NH—COR², halogen, C≡CR², —C═CR²₂, or —C═CHR, in which each R² independently represents a hydrogen atom or alkyl, aryl, or alkyl-aryl group.

2. The compound of claim 1, wherein L represents —SR³, —SO₂R³, —OSO₂R³, —N⁺R³₃, —N⁺HR³₂, —N⁺H₂R³, halogen, or —O◯, in which R³ represents a hydrogen atom or an alkyl, aryl, or alkyl-aryl, and ◯ represents a substituted aryl group containing at least one electron withdrawing substituent.

3. The compound of claim 1, wherein V represents —(CH₂)ₙSO₃X, in which n=1-6, and X represents H or counterion.

4. The compound of claim 1, wherein V represents a hydrophilic polymer.

5. The compound of claim 1, wherein X represents a drug, a diagnostic moiety, or a chelating agent.

6. The compound of claim 1, wherein X represents a detection moiety or hapten.

7. The compound of claim 1, wherein X represents a protein.

8. A kit comprising the compound of claim 1.

9. A conjugate of the general formula (2) 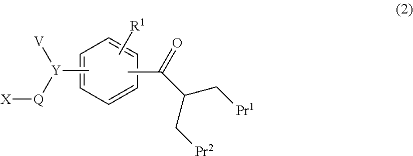
wherein:
X represents a payload;
Q represents a cleavable or non-cleavable linking group;
Y represents an amide group;
V represents a moiety that improves cross-linking and/or labeling efficiency;
R¹ represents H, alkyl, —CN, —NO₂, —CO₂R, —COH, —CH₂OH, —COR², —OR², —OCOR², —OCO₂R², —SR², —SO, —SO₂R², —NHCOR², —NR²COR², —NHCO₂R², —NO, —NHOH, —NR²OH, —C═N—NH—COR², halogen, —C≡CR², —C═CR²₂, or —C═CHR, in which each R² independently represents a hydrogen atom or alkyl, aryl, or alkyl-aryl group;
both Pr¹ or Pr² together represent a single protein or peptide bonded at two separate points via two thiol groups generated by the reduction of a disulfide bridge in the protein.

10. The conjugate of claim 9, wherein X represents a drug, a diagnostic moiety, or a chelating agent.

11. The conjugate of claim 9, wherein V represents a hydrophilic polymer.

12. The conjugate of claim 9, wherein V represents a homo- or co-polymer selected from the group consisting of polyalkylene glycols, polyvinylpyrrolidones, polyacrylates, polymethacrylates, polyoxazolines, polyvinylalcohols, polyacrylamides, polymethacrylamides, HPMA copolymers, polyesters, polyacetals, poly(ortho ester)s, polycarbonates, poly(imino carbonate)s, polyamides, copolymers of divinylether-maleic anhydride and styrene-maleic anhydride, polysacoharides, and polyglutamic acids.

13. The conjugate of claim 9, wherein V represents discrete or non-discrete, branched or linear polyethylene.

14. The conjugate of claim 9, wherein V represents —(CH₂)ₙSO₃X, in which n=1-6, and X represents H or counterion.

15. The conjugate of claim 9, wherein both Pr¹ and Pr² together represent a single full length antibody or an antibody fragment comprising an antigen-binding region of the full length antibody.

16. A compound of the general formula (3): 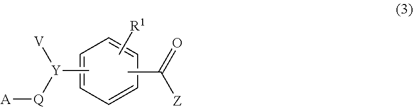
wherein:
Q represents a cleavable or non-cleavable linking group;
Y represents an amide group;
V represents a moiety that improves cross-linking and/or labeling efficiency or is absent;
Z represents either —CH—(CH₂L)₂ or —C(CH₂L)(═CH₂);
L independently represents a leaving group;
R¹ represents H, alkyl, —CN, —NO₂, CO₂R, —COH, —CH₂OH, —COR², —OR², —OCOR², —OCO₂R², —SR², —SO, —SO₂R², —NHCOR², —NR²COR², —NHCO₂R², —NO, —NHOH, —NR²OH, —C═N—NH—COR², halogen, —C≡CR², —C═CR²₂, or —C═CHR, in which each R² independently represents a hydrogen atom or alkyl, aryl, or alkyl-aryl group; and
A represents a reactive moiety partner of a pair of orthogonally reactive moieties that can react with each other without activation and both reactive moieties are sufficiently stable under commonly applied protein labeling conditions.

17. The compound of claim 16, wherein L represents —SR³, —SO₂R³, —OSO₂R³, —N⁺R³₃, —N⁺HR³₂, —N⁺H₂R³, halogen, or —O◯, in which R³ represents a hydrogen atom or an alkyl, aryl, or alkyl-aryl, and ◯ represents a substituted aryl group containing at least one electron withdrawing substituent.

18. The compound of claim 16, wherein V represents H.

19. The compound of claim 16, wherein V represents —(CH₂)ₙSO₃X, in which n=1-6, X represents H or counterion.

20. The compound of claim 16, wherein V represents a hydrophilic polymer.

21. The compound of claim 16, wherein A is selected from the group consisting of orthogonal reactive pairs that undergo Staudinger ligation, strain-promoted Huisgen 1,3-cycloaddition, Inverse Demand Diels-Alder cycloaddition, and hydrazone or oxime bond forming reactions.

22. The compound of claim 16, wherein A represents a 1,3-dipole group.

23. The compound of claim 22, wherein the 1,3-dipole group is selected from the group consisting of an azide, a nitrile oxide, a nitrone, an azoxy group, and an acyl diazo group.

24. The compound of claim 16, wherein A represents a substituted or unsubstituted cyclooctyne that undergoes a 1,3-cycloadditon reaction with a 1,3 dipole group.

25. The compound of claim 16, wherein A represents a diene that undergoes an Inverse electron-demand Diels-Alder reaction.

26. The compound of claim 25, wherein the diene that undergoes an Inverse electron-demand Diels-Alder reaction is a substituted or unsubstituted tetrazine.

27. The compound of claim 16, wherein A represents a dienophile that undergoes Inverse electron-demand Diels-Alder reaction.

28. The compound of claim 27, wherein the dienophile that undergoes Inverse electron-demand Diels-Alder reaction is a substituted or unsubstituted trans-cyclooctene, norbornene, cyclopropene, or N-acylazetine.

29. A method for the preparation of a protein conjugate of the general formula (4): 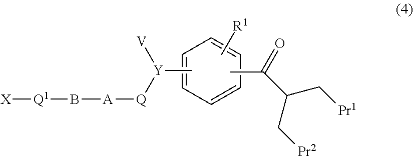
wherein:
X represents a payload;
Q and Q¹ independently represent a cleavable or non-cleavable linking group;
Y represents an amide group;
V represents a moiety that improves cross-linking and/or labeling efficiency or is absent;
R¹ represents H, alkyl, —CN, —NO₂, —CO₂R, —COH, —CH₂OH, —COR², —OR², —OCOR², —OCO₂R², —SR², —SO, —SO₂R², —NHCOR², —NR²COR², —NHCO₂R², —NO, —NHOH, —NR²OH, —C═N—NH—COR², halogen, —C≡CR², —C═CR²₂, or —C═CHR, in which each R² independently represents a hydrogen atom or alkyl, aryl, or alkyl-aryl group;
both Pr¹ or Pr² together represent a single protein or peptide bonded at two separate points via two thiol groups generated by the reduction of a disulfide bridge in the protein;
A-B is a pair of orthogonally reactive moieties that can react with each other without activation and are stable under commonly applied protein labeling conditions;
said method comprising the steps of:
(a) reducing a disulfide bridge in the protein; and
(b) reacting the reduced protein with a compound of general formula (3) to form an activated protein of general formula (5): 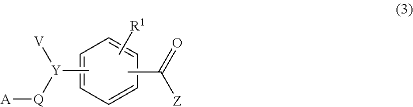
wherein Z represents either —CH—(CH₂L)₂ or —C(CH₂L)(═CH₂), L independently represents a leaving group; 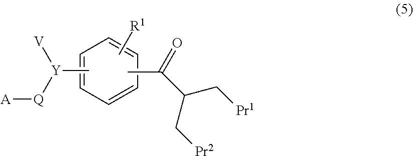
(c) reacting said activated protein with a compound of general formula (6):
X-Q¹-B (6).

30. The method of claim 29, wherein L represents —SR³, —SO₂R³, —OSO₂R³, —N⁺R³₃, —N⁺HR³₂, —N⁺H₂R³, halogen, or —O◯, in which R³ represents a hydrogen atom or an alkyl, aryl, or alkyl-aryl, and ◯ represents a substituted aryl group containing at least one electron withdrawing substituent.

31. The method of claim 29, wherein V represents H.

32. The method of claim 29, wherein V represents —(CH₂)ₙSO₃X, in which n=1-6, and X represents H or counterion.

33. The method of claim 29, wherein V represents a hydrophilic polymer.

34. The method of claim 29, wherein A is selected from the group consisting of orthogonal reactive pairs that undergo Staudinger ligation, strain-promoted Huisgen 1,3-cycloaddition, Inverse Demand Diels-Alder cycloaddition, and hydrazone or oxime bond forming reactions.

35. The method of claim 29, wherein A represents a 1,3-dipole group.

36. The method of claim 35, wherein the 1,3-dipole group is selected from the group consisting of an azide, a nitrile oxide, a nitrone, an azoxy group, and an acyl diazo group.

37. The method of claim 29, wherein A represents a substituted or unsubstituted cyclooctyne that undergoes a 1,3-cycloadditon reaction with a 1,3 dipole group.

38. The method of claim 29, wherein A represents a diene that undergoes an Inverse electron-demand Diels-Alder reaction.

39. The method of claim 38, wherein the diene that undergoes an Inverse electron-demand Diels-Alder reaction is a substituted or unsubstituted tetrazine.

40. The method of claim 29, wherein A represents a dienophile that undergoes an Inverse electron-demand Diels-Alder reaction.

41. The method of claim 40, wherein the dienophile that undergoes Inverse electron-demand Diels-Alder reaction is a substituted or unsubstituted trans-cyclooctene, norbornene, cyclopropene, or N-acylazetine.

42. The method of claim 29, wherein X represents a drug, a diagnostic moiety, or a chelating agent.

43. The method of claim 29, wherein X represents a biopolymer or a synthetic polymer.

44. The method of claim 43, wherein the biopolymer is a protein.

45. The method of claim 43, wherein the synthetic polymer is PEG.
